(19) United States
(12) Patent Application Publication
(10) Pub. No.: US 2011/0230430 A1
(43) Pub. Date: Sep. 22, 2011

(54) SAPONIN EXTRACT FROM SAPONARIA SPP. AND USES THEREOF

(75) Inventors: Robert Hickie, Saskatoon (CA); John Balsevich, Saskatoon (CA); Irving Ramirez-Erosa, Saskatoon (CA); Donna Dunlop, Clavet (CA); Greg Bishop, Saskatoon (CA); Leah Deibert, Saskatoon (CA); Paul Arnison, Saskatoon (CA)

(73) Assignee: NATIONAL RESEARCH COUNCIL OF CANADA, Ottawa (CA)

(21) Appl. No.: 12/934,677

(22) PCT Filed: Mar. 26, 2009
(86) PCT No.: PCT/CA2009/000394

§ 371 Date: May 19, 2011

Related U.S. Application Data
(60) Provisional application No. 61/070,796, filed on Mar. 26, 2008.

(51) Int. Cl. A61K 31/7028; C07H 15/256; A61P 35/00; C12N 5/00

(52) U.S. Cl. 514/33; 536/18.1; 435/375

(57) ABSTRACT

A saponin extract from Saponaria vaccaria and its use for stimulating apoptosis in cancer cells and treating cancer is described. The saponin extract may be isolated from Saponaria vaccaria seed. The saponin extract may comprise one or more than one triterpene saponin. The triterpene saponin may comprise a bisdesmosidic saponin having a molecular weight selected from the group consisting of molecular weight 1448, 1464, 1422, 1526, 1596 and 1688. Bisdesmosidic saponin of molecular weight 1448, 1464, 1422, 1526, 1596 and 1688 isolated from Saponaria vaccaria seed may be used to treat human cancer including prostate cancer, human breast cancer and colon cancer. Also disclosed is a method of isolating the saponin extract.

### FIELD OF INVENTION

The present invention relates to a saponin extract from Saponaria spp. The present invention also relates to a triterpene saponin, and to methods of isolating a triterpene saponin from Saponaria spp. The present invention also provides uses of the triterpene saponin obtained from Saponaria spp.

### BACKGROUND OF THE INVENTION

Saponaria, also known as soapwort, is a genus of about 20 species of largely perennial herbs in the Caryophyllaceae, native to southern Europe, North Africa and southwest Asia. Saponaria vaccaria commonly referred to as cow cockle, spring cockle, pink cockle and China cockle, is an annual weed commonly found in grain fields of northwestern United States and in the prairie provinces of Canada, having been introduced originally from Europe. The Saponaria vaccaria (also known as Vaccaria segetalis, Vaccaria hispanica and Vaccaria pyramidata) seed is rich in saponins. Saponaria officinalis has been used for medicinal studies and is grown in Europe as a perennial ornamental. This species is well known as a source of saponins and is known as a soapwort in Europe.

Saponins are high molecular complexes of sugars attached to a central terpenoid or steroid aglycone core. The structures are very diverse but they have a common chemical property which is the ability to interact with both hydrophilic and hydrophobic substances. This amphipathic property makes saponins natural detergents and foaming agents and leads directly to biological activity through interaction with membranes.

Saponins are made by many taxonomically diverse species of plants and these substances are thought to be part of the plants natural biochemical defense system against pests and pathogens. Saponins have agricultural applications that include activity against fungi, bacteria and nematodes. Saponins also have a wide range of human health care, therapeutic and medicinal applications (Francis et al. 2002; Sparg et al., 2004). The tritrepenoid saponins, for example, from the Soap bark tree, Quillaja saponaria, have immuno-stimulatory properties and when combined with steroids such as cholesterol have been investigated extensively as adjuvants for vaccine formulation. Additionally, plant extracts comprising saponins, especially from legumes such as soybean and alfalfa, are known to bind to and lower blood cholesterol. Other health care applications for saponins include treatment of dementia, treatment of depression, treatment of premenstrual syndrome, antibiotic, anti-bacterial, anti-viral, anti-fungal, anti-inflammatory, anti-convulsant, treatment of diabetes, and treatment of obesity.

Saponins from different sources have been found to inhibit cell division, and stimulate the natural cell death cascade of apoptosis. For example, triterpenoid saponins from hairy root cultures of Acacia victorae have been described to stimulate apoptosis (U.S. Pat. No. 6,444,233; U.S. Pat. No. 6,746,696; U.S. Pat. No. 7,105,186; and U.S. Pat. No. 6,689,398). Farming of Acacia victorae on a scale large enough for isolation of triterpeniod saponins as a useful pharmaceutical compound is prohibitally expensive. Further, the triterpeniods saponins are isolated from the hairy root cultures of the tree. These saponins will typically be different from the saponins made by the plant and will possess different bioactivities.

Activation of apoptosis by anticancer drugs is a key mechanism of action for anti-tumor drugs. Apoptosis is a process in which a cell actively terminates itself by the destruction of vital cell components or DNA, via various molecular signaling pathways. In apoptosis the cell condenses and fragments into membrane bound apoptotic bodies, which are ingested and destroyed by the immune system.

Apoptosis is an active process that requires cellular energy. This is in contrast to necrosis, which is cell death due to injury or stress. Apoptosis results from a complex cascade of destructive cellular processes mediated by enzymes called caspases (cysteinyl aspartate specific proteinases). Fourteen different caspases have been identified, they exist as inactive crystalline zymogens that are activated during apoptosis. Caspase enzymes further activate other caspase enzymes in a coordinated cascade. Cellular changes that occur include activation of DNAse activity and degradation of DNA, loss of cytoskeletal proteins and resultant loss of cell shape and integrity.

Stimulation of apoptosis is known to occur via two main pathways, (Debatin, 1999; and Kaufmann et al., 2000) The first apoptosis pathway is the death receptor pathway involving for example Fas and other members of the tumor necrosis factor receptor family that activate caspase-8. Caspase-8 activates further down stream events including caspase-3 or cleavage of Bid. The second apoptosis pathway is a mitochondrial pathway activated when factors such as cyctochrome C are released. Released cytochrome C interacts with Apaf-1 and activates caspase-9 that in turn activates caspase-3, a principle protease involved in apoptosis. (Hengartner, 2000; Kroemer et al., 2000; and Gulbins et al., 2003).

Plant species that produce saponins or related triterpenoid compounds that have been ascribed potential anti-cancer activity include:
Anenome raddeana (US 2004/006763)
Albizia adianthifolia (Haddad, M., 2004)
Aralia dasyphylla (Xiao et al., 1999)
Aster lingulatus (Shao, et al., 1997)
Astragalus sp. (Yesiladaa et al., 2005; Lin et al., 2003)
Black bean (US 20060024394)
Birch (Cichewicz, et al., 2004; Atopinka, et al., 1999)
Bolbostemma paniculatum (Wang, et al., 2006)
Bupleurum sp. (Hsu, et al., 2000; Hsu, et al., 2004)
Clematis chinensis (Mimaki, et al., 2004)
Cleome Africana (Nagaya, et al., 1997)
Digitalis purpurea (Lin et al., 2004; López-Lāzaro, et al., 2003; López-Lāzaro, et al., 2005)
Dysoxylum cumingianum (Kashiwada, et al., 1997)
Enterolobium contortisiliquum (Mimaki, et al., 2003)
Ficus microcarpa (Chaing, et al., 2005)
Ginseng (U.S. Pat. No. 5,919,770; Huang et al., 2004; U.S. Pat. No. 6,888,014; U.S. Pat. No. 6,949,523; Hwang, et al., 2002; Wargovich, 2001; Kim et al., 2004; Atopinka, et al., 1999; Chang, et al., 2003)
Gleditsia sinensis (Chow, et al., 2002; Zhong, et al., 2004)
Gypsophilia sp. (Sung, et al., 1995)
Hedera colchica (Barthomeuf, et al., 2004)
Ixeris sonchifolia (Feng, et al, 2003)
Ludwigia octovalis (Chang, et al., 2004)
Lysimachia davurica (Tian, et al., 2006)
Platycodon grandiflorum (Park, et al., 2004)
Polyscias amplifolia (Prakash Chaturvedula, et al., 2003)
Quillaja saponaria (Soapbark tree) (Wang, et al., 2006; US 2005/0175623; Wu, et al., 2004)
Reissantia buchananii (Wu, et al., 2005)
Sanguisorba officinalis (Mimaki, et al., 2001)
Schefflera fagueti (Cloffi, et al., 2003)
Securidaca inappendiculata (Yui, et al., 2001; Yui, et al., 2003)
Silene sp. (Gaidi, et al., 2002)
Solidago virgaurea (Plohmann, et al., 1997; Gross, et al., 2002)
Soybean (U.S. Pat. No.6,900,240; Yanamandra, et al., 2003; Ellington, et al., 2005)
Strophanthus gratus (Huang, et al., 2004)
Viguiera decurrens (Marquina, et al., 2001)
Xanthoceras sorbifolia (US 2005/0276872)

Although a growing number of reports have identified saponins from a wide variety of sources, with a diverse array of physiological properties, there is still a need to identify new and unique activities of significant medical importance. There is no previous disclosure that saponins from Saponaria vaccaria may cause apoptosis of cancer cells and in particular no disclosure that a triterpenoid saponin isolated from the species Saponaria vaccaria may be useful for preventing and treating cancers.

Even though saponins can have potentially useful therapeutic activities they can also have toxic side effects to normal cells or they can be haemolytic and disrupt blood cell membranes due to detergent activity (Wang et al, 2007). Additionally, saponins occur naturally in an array of complex mixtures of closely related structures that are difficult to separate, and individual saponins within these complex mixtures may have entirely different or opposite activities. The great diversity of different, closely related forms, creates difficulties for their effective use as therapeutic agents. This issue is compounded by the practical aspects of production and preparation of materials on a cost effective commercial scale as many of the species either make low concentrations of the active substance, the species itself is not convenient for agricultural production or the saponin resides in tissues and materials that are not easily processed.

Although saponins are produced by many species of plants, including some common cultivated crops, saponins with significant apoptosis inducing activity may be highly expensive to produce if the species in question is rare, difficult to cultivate, difficult to process or difficult to purify to clinically useful concentrations. Saponins are typically produced in a large array of closely related forms that are difficult to separate and hence expensive to purify. Saponins can be used as complex mixtures however individual saponins with slightly different physical structure may have highly dissimilar biological activity.

All of these factors combined lead to the need to find saponins of medicinal value that can be cultivated and isolated in a convenient cost effective commercial scale from plant species amenable to large scale conventional agricultural production.

### SUMMARY OF THE INVENTION

The present invention relates to a saponin extract from Saponaria spp. The present invention also relates to a triterpene saponin isolated from Saponaria spp, and to methods of isolating a triterpene saponin from Saponaria spp. The present invention also provided uses of the triterpene saponin obtained from Saponaria spp.

The present invention provides novel saponin compounds and mixtures isolated form seed of Saponaria spp. for example, Saponaria vaccaria and methods for their use. The saponins disclosed herein comprise a triterpenoid agylcone to which are attached sugars, acyl and other chemical moieties that would be apparent to one skilled in the art. The triterpene saponins of the present invention typically have molecular weights in the range 1300 to 1900. Preferably, the triterpenoid aglycone is of the quillajic acid type structure and has the ability to induce apoptosis in PC-3 prostate cancer cells, MDA-MB-231 and MCF-7 breast cancer cells, HT-29 and WiDr colon cancer cells at less than 10 μM concentration, for example from about 0.01 μM to about 10 μM.

According to the present invention there is provided a saponin extract from Saponaria spp. The Saponaria spp. may be Saponaria vaccaria, and the saponin extract may be isolated from Saponaria vaccaria seed. The saponin extract may comprise one or more than one triterpene saponin. The triterpene saponin may comprise a bisdesmosidic saponin having a molecular weight selected from the group consisting of molecular weight 1448, 1464, 1422, 1526, 1596, 1688 and a combination thereof. The triterpene saponin may comprise a bisdesmosidic saponin having molecular weight 1448. The triterpene saponin may comprise a bisdesmosidic saponin having molecular weight 1464. The triterpene saponin may comprise a bisdesmosidic saponin having molecular weight 1422. The triterpene saponin may comprise a bisdesmosidic saponin having molecular weight 1526. The triterpene saponin may comprise a bisdesmosidic saponin having molecular weight 1596. The triterpene saponin may comprise a bisdesmosidic saponin having molecular weight 1688.

The present invention also provides a method of stimulating apoptosis in cancer cells, comprising treating the cancer cells with the saponin extract of the present invention.

The present invention further provides a method of stimulating apoptosis in cancer cells, comprising treating the cancer cells with a saponin extract from Saponaria vaccaria comprising one or more than one bisdesmosidic saponin of molecular weight 1448, 1464, 1422, 1526, 1596, 1688 and a combination thereof.

The present invention further provides a method of stimulating apoptosis in cancer cells, comprising treating the cancer cells with a Saponaria vaccaria bisdesmosidic saponin having a molecular weight selected from the group consisting of molecular weight 1448, 1464, 1422, 1526, 1596, 1688 and a combination thereof. The bisdesmosidic saponin may have molecular weight 1448. The bisdesmosidic saponin may have molecular weight 1464. The bisdesmosidic saponin may have molecular weight 1422. The bisdesmosidic saponin may have molecular weight 1526. The bisdesmosidic saponin may have molecular weight 1596. The bisdesmosidic saponin may have a molecular weight 1688.

The present invention provides a pharmaceutical composition comprising a therapeutically effective amount of the saponin extract of the present invention.

The present invention also provides a pharmaceutical composition comprising a therapeutically effective amount of a saponin extract from Saponaria vaccaria comprising one or more than one bisdesmosidic saponin of molecular weight 1448, 1464, 1422, 1526, 1596, 1688 and a combination thereof, and a pharmaceutically acceptable carrier.

The present invention further provides a pharmaceutical composition comprising a therapeutically effective amount of a Saponaria vaccaria bisdesmosidic saponin having a molecular weight selected from the group consisting of molecular weight 1448, 1464, 1422, 1526, 1596, 1688 and a combination thereof, and a pharmaceutically acceptable carrier. The bisdesmosidic saponin may have molecular weight 1448. The bisdesmosidic saponin may have molecular weight 1464. The bisdesmosidic saponin may have molecular weight 1422. The bisdesmosidic saponin may have molecular weight 1526. The bisdesmosidic saponin may have molecular weight 1596. The bisdesmosidic sapponin may have a molecular weight 1688.

The present invention provides a method of treating a subject with cancer comprising administering the pharmaceutical composition of the present invention to the subject. The cancer may be prostrate cancer or breast cancer or colon cancer.

The present invention provides use of the pharmaceutical composition of the present invention for treating cancer. The cancer may be prostrate cancer or breast cancer or colon cancer.

The present invention provides a method of preparing an isolated saponin extract from Saponaria vaccaria with apoptosis stimulating activity (method A) comprising: a) milling seed of Saponaria vaccaria; and b) treating the milled seed with a solvent to produce the saponin extract.

The present invention also provides a method (Method A) of preparing an isolated bisdesmosidic saponin composition comprising at least 45% bisdesmosidic saponins comprising: a) milling seed of Saponaria vaccaria, b) treating the milled seed with a solvent to produce a saponin extract, c) drying the saponin extract to produce a crude saponin composition, d) titurating the crude saponin composition with methanol, and e) recovering a solid residue comprising at least 45%, by weight, of bisdesmosidic saponins.

The present invention further provides a method of preparing an isolated bisdesmosidic saponin composition from Saponaria vaccaria comprising at least 60% by weight bisdesmosidic saponins with apoptosis stimulating activity (method B), the method comprising: a) milling seed of Saponaria vaccaria; b) treating the milled seed with a solvent to produce a saponin mixture; c) cooling the saponin mixture to precipitate bisdesmosidic saponins; and d) recovering the precipitate comprising the isolated bisdesmosidic saponin composition. The solvent may be aqueous methanol, such as but not limited to 70% aqueous methanol. The saponin mixture may be stored at a temperature of at least minus 20 degrees C. for about 36 to about 60 hours or any amount of time therebetween. The step of recovering the precipitate (step d) may be carried out by decantation and reduction to dryness in vacuo to produce a solid residue comprising the bisdesmosidic saponin composition. The method may further comprise addition of a second solvent to the saponin mixture following step (b). The second solvent may be ethanol. The amount of ethanol added to the saponin mixture, may be sufficient to produce an extract comprising 40-50% by volume ethanol, or any amount therebetween.

The present invention pertains to the method described above (method B) further comprising: e) applying the isolated bisdesmosidic saponin composition to a column to effect separation of individual saponins; and f) recovering bisdesmosidic saponin of molecular weight 1448. The bisdesmosidic saponin of molecular weight 1448 may be recovered in step f) by elution of the column with 100% methanol.

The present invention pertains to the method described above (method B) further comprising: e) applying the isolated bisdesmosidic saponin composition to a column to effect separation of individual saponins; and f) recovering bisdesmosidic saponin of molecular weight 1464. The bisdesmosidic saponin of molecular weight 1464 may be recovered in step f) by elution of the column with at least 80% methanol.

The present invention pertains to the method described above (method B) further comprising: e) applying the isolated bisdesmosidic saponin composition to a column to effect separation of individual saponins; and f) recovering bisdesmosidic saponin of molecular weight 1422. The bisdesmosidic saponin of molecular weight 1422 may be recovered in step f) by dissolving bisdesmosidic saponin of molecular weight 1422 in a solvent and treating with a base to obtain a basic composition, applying the basic composition to a column and eluting with 75% methanol.

The present invention pertains to the method described above (method B) further comprising: e) applying the isolated bisdesmosidic saponin composition to a column to effect separation of individual saponins; and f) recovering bisdesmosidic saponin of molecular weight 1526. The bisdesmosidic saponin of molecular weight 1526 may be recovered in step f) by elution of the column with at 70-75% methanol.

The present invention pertains to the method described above (method B) further comprising: e) applying the isolated bisdesmosidic saponin composition to a column to effect separation of individual saponins; and f) recovering bisdesmosidic saponin of molecular weight 1596.

The present invention also provides a method of preparing an isolated saponin extract from Saponaria vaccaria comprising bisdesmosidic saponin of molecular weight 1448 (method C), the method comprising: a) milling seed of Saponaria vaccaria; b) treating the milled seed with a solvent thereby extracting a saponin composition; c) applying the saponin composition to a column to effect separation of individual saponins; and d) recovering the bisdesmosidic saponin of molecular weight 1448. The bisdesmosidic saponin of molecular weight 1448 may be recovered in step d) by elution of the column with 100% methanol.

The present invention also provides a method of preparing an isolated saponin extract from Saponaria vaccaria comprising bisdesmosidic saponin of molecular weight 1464 (method D), the method comprising: a) milling seed of Saponaria vaccaria; b) treating the milled seed with a solvent thereby extracting a saponin composition; c) applying the saponin composition to a column to effect separation of individual saponins; and d) recovering the bisdesmosidic saponin of molecular weight 1464. The bisdesmosidic saponin of molecular weight 1464 may be recovered in step d) by elution of the column with at least 80% methanol.

The present invention also provides a method of preparing an isolated saponin extract from Saponaria vaccaria comprising bisdesmosidic saponin of molecular weight 1596 (method E), the method comprising preferentially isolated from a variety which contains a high titre of 3-O-trisachharide bisdesmosidics in seeds (for example Scott variety; Balsevich et al, Phytochemical Analysis) or from roots, by chromatography on reverse phase media, using methanol-water gradients and by chromatography on reverse phase media, using methanol-water-acetonitrile gradients.

The present invention also provides a method of preparing an isolated saponin extract from Saponaria vaccaria comprising bisdesmosidic saponin of molecular weight 1596 (method F), the method comprising: a) drying and pulverizing roots from Saponaria vaccaria; b) treating the pulverized roots with a solvent thereby extracting a saponin composition; c) applying the saponin composition to a column to effect separation of individual saponins; and d) recovering the bisdesmosidic saponin of molecular weight 1596. The bisdesmosidic saponin of molecular weight 1596 may be recovered in step d) by elution of the column with 80% methanol.

The present invention also provides a method of preparing an isolated saponin extract from Saponaria vaccaria comprising bisdesmosidic saponin of molecular weight 1526 (method G), the method comprising: a) milling seed of Saponaria vaccaria; b) treating the milled seed with a solvent thereby extracting a saponin composition; c) applying the saponin composition to a column to effect separation of individual saponins; and d) recovering the bisdesmosidic saponin of molecular weight 1526. The bisdesmosidic saponin of molecular weight 1526 may be recovered in step d) by elution of the column with at least 70-75% methanol.

The present invention also provides a method of preparing an isolated saponin extract from Saponaria vaccaria comprising bisdesmosidic saponin of molecular weight 1422 (method H), the method comprising: a) milling seed of Saponaria vaccaria; b) treating the milled seed with a solvent thereby extracting a saponin composition; c) applying the saponin composition to a column to effect separation of individual saponins; d) recovering bisdesmosidic saponin of molecular weight 1464; e) dissolving the bisdesmosidic saponin of molecular weight 1464 in a solvent and treating with a base to obtain a basic composition; f) applying the basic composition to a column to effect separation of individual saponins; and g) recovering the bisdesmosidic saponin of molecular weight 1422. The bisdesmosidic saponin of molecular weight 1464 may be recovered in step d) by elution of the column with at least 80% methanol. The bisdesmosidic saponin of molecular weight 1422 may be recovered in step g) by elution of the column with 75% methanol. The solvent in step e) may be methanol, such as 30% methanol. The base in step e) may be an hydroxide such as ammonium hydroxide.

The present invention provides a method of preparing an isolated saponin extract from Saponaria vaccaria comprising bisdesmosidic saponin of molecular weight 1688 (isomer 1), the method (method I) comprising: a) drying and pulverizing roots from Saponaria vaccaria; b) treating the pulverized roots with a solvent thereby extracting a saponin composition; c) applying the saponin composition to a column to effect separation of individual saponins; d) recovering the bisdesmosidic saponin of molecular weight 1772; e) dissolving the bisdesmosidic saponin of molecular weight 1772 in a solvent and treating with a base to affect deacetylation to produce a deacteylated composition; and f) applying the deacetylated composition to a column to affect separation of saponins. The bisdesmosidic saponin 1772 may be recovered in step d) from the column typically by elution with 80-85% methanol. The bisdesmosidic saponin molecular weight 1688 (isomer 1) may be recovered in step f) by elution with 65% methanol. The base in step e) may be an hydroxide such as ammonium hydroxide.

The present invention provides a method of preparing an isolated saponin extract from Saponaria vaccaria comprising bisdesmosidic saponins of molecular weight 1688 (isomers 1 and 2), the method (method J) comprising: a) milling seed of Saponaria vaccaria; b) treating the milled seed with a solvent thereby extracting a saponin composition; c) dissolving the saponin mixture in a solvent and treating the solution with base to affect deacetylation; and d) separating individual saponins on a column and recovering 1688-1 and 1688-2. The bisdesmosidic saponins may be recovered in step d) by elution with 65-70% methanol.

The present invention also envisions a method of inducing apoptosis in a malignant mammalian cell in a mammal comprising, administering to the mammal a therapeutically effective amount of the pharmaceutical compositions described herein. The cell may be a skin cell, a colon cell, a uterine cell, an ovarian cell, a pancreatic cell, a lung cell, a bladder cell, a prostate cell, a renal cell, a breast cell, or a combination thereof.

This summary of the invention does not necessarily describe all features of the invention.

An advantage of the present invention is that Saponaria vaccaria can be manufactured on a large scale at reasonable costs using conventional farming practices and machinery. The biologically active triterpene saponin extract can also be isolated from Saponaria vaccaria on a large scale at reasonable cost using the method of the present invention.

### BRIEF DESCRIPTION OF THE DRAWINGS

These and other features of the invention will become more apparent from the following description in which reference is made to the appended drawings wherein:

FIG. 1 is a schematic representation of preparation of various saponin containing extracts from S. vaccaria seed and roots by extraction and processing.;

FIG. 2A is a schematic representation of isolation or preparation of saponins PC1526, 1464, 1448, 1422 and 1380. FIG. 2B is a schematic representation of isolation or preparation of saponin1596, 1772, 1688-1 and 1688-2 from crude root extract.

FIG. 3 shows a family tree of quillaic acid bisdesmosidic saponin fragments isolated from Saponaria vaccaria seed. FIG. 3A shows Type I, 3-O disaccharides. FIG. 3B shows Type I, 3-O trisaccharides. FIG. 3C shows Type II, 3-O disaccharides. FIG. 3D shows Type II, 3-O trisaccharides. FIG. 3E shows Type III, 3-O disaccharides. FIG. 3F shows Type III, 3-O trisaccharides. Legend: QA=quillaic acid, GLUR=glucopyranosiduronic acid, Gal=galactopyranose, XYL=xylopyranose, FUC=fucopyranose, ARA=arabinofuranose, RHA=rhamnopyranose, GLC=glucopyranose, Ac=acetate,

FIG. 4 shows nuclei of cells that have been treated with various saponin extracts. FIG. 4A shows fluorescent microscopy of Hoechst 33342 stained nuclei in untreated CRL-2522 fibroblast cells (upper panel) and CRL-2522 fibroblast cells treated with 14 μM PC1448 saponin extract (lower panel). FIG. 4B shows fluorescent microscopy of Hoechst 33342 stained nuclei in untreated PC-3 cells (upper panel) and PC-3 cells treated with 14 μM PC1448 saponin extract (lower panel). FIG. 4C shows fluorescent microscopy of Hoechst 33342 stained nuclei in untreated MDA-MB-231 cells (upper panel) and MDA-MB-231 cells treated with 14 μM PC1448 saponin extract (lower panel). Arrows in FIGS. 4A, 4B and 4C indicate nuclear changes characteristic of apoptotic cells;

FIG. 5 shows a comparison of potencies of PC1526, 1422, and 1448 saponins on HT-29 Cancer Cells (Human Colon Cancer Cells) using the Dual Sensor: MitoCasp™ Assay

FIG. 6 shows the results of the Dual Sensor: MitoCasp™ Assay for HT-29 Colon Cancer Cells treated with PC1380.

FIG. 7 shows the results of the Dual Sensor: MitoCasp™ Assay for WiDr Colon Cancer Cells treated with PC1526.

FIG. 8 shows the results of the Dual Sensor: MitoCasp™ Assay for WiDr Colon Cancer Cells treated with PC1448.

FIG. 9 shows the results of the Dual Sensor: MitoCasp™ Assay for WiDr Colon Cancer Cells treated with PC1596.

FIG. 10 shows the results of the Dual Sensor: MitoCasp™ Assay for MDA-MB-231 Breast Cancer Cells treated with Calendula saponin.

FIG. 11 shows the result of the Dual Sensor: MitoCasp™ Assay for PC-3 Prostate Cancer Cells treated with PC1688-1 (1688 isomer 1).

FIG. 12 shows the results of the Dual Sensor: MitoCasp™ Assay for PC-3 Prostate Cancer Cells treated with PC1688-2 (1688 isomer 2).

FIG. 13 shows the results of the Dual Sensor: MitoCasp™ Assay for MDA-MB-231 Breast Cancer Cells treated with PC1688-1.

FIG. 14 shows the results of the Vybrant® Apoptosis Assay Kit #2 for PC-3 Prostate Cancer Cells treated with PC1526.

FIG. 15 shows the results of the Vybrant® Apoptosis Assay Kit #2 for MDA-MB-231 Cancer Cells treated with PC1526.

FIG. 16 shows the results of the Dual Sensor: MitoCasp™ Assay for MCF-7 Breast Cancer Cells treated with PC1526.

### DETAILED DESCRIPTION

The present invention relates to a saponin extract and individual saponins from Saponaria spp. and uses thereof.

The following description is of a preferred embodiment.

According to the present invention there is provided a saponin extract from Saponaria spp. For example, the saponin may be isolated from Saponaria vaccaria seed. The saponin extract may comprise one or more than one triterpene saponin, for example but not limited to, a bisdesmosidic saponin having a molecular weight selected from about 1300 to about 1900, and may be selected from the group consisting of molecular weight 1448, 1464, 1422, 1506, 1526, 1596, 1688 and a combination thereof. The use of mixture of triterpene saponins as disclosed herein is also contemplated. Preferably, the triterpenoid aglycone is of the quillajic acid type structure and has the ability to induce apoptosis in PC-3 prostate cancer cells, MDA-MB-231 breast cancer cells, MCF-7 breast cancer cells, HT-29 colon cancer cells and WiDr Colon Cancer Cells at a 7-14 μM (or less than 14 μM) concentration as described below.

The saponin extract may be collected from Saponaria vaccaria tissues, for example, the seed, by various means as described herein. The tissues used in this process may comprise seeds or roots, however leaves, stems, seedlings, or mixtures thereof may also be used. The tissue, for example the seed or root, may be defatted and the defatted meal extracted with any organic solvent which is capable of extracting, often by dissolving, the saponin compound of interest. Useful extraction solvents are methanol, ethanol, isopropyl alcohol, dichloromethane, chloroform, ethyl acetate, water, glycerol and mixtures thereof.

The saponin extract may also be prepared using a tissue culture comprising cells of a Saponaria vaccaria plant; and extracting the triterpene saponin composition from the cells with a solvent. The tissue culture may comprise a hairy root culture, or the tissue culture may be a cell suspension culture.

The saponin extract may comprise one or more than one triterpene saponin. Triterpene or triterpene glycoside refers to biologically active saponin compounds identified herein from Saponaria spp. for example, Saponaria vaccaria. The triterpene or triterpene glycosides need not be isolated from Saponaria vaccaria only, as one of skill in the art, in light of the present disclosure, could isolate the compounds from a related species, or chemically synthesize analogs of the triterpenes and triterpene glycosides disclosed herein. “Triterpenes” of this invention include the saponin compounds described herein which have at least a triterpene unit(s) and, in the case of triterpene glycosides, a sugar or saccharide. Triterpene saponins may also have additional moieties or chemical functionalities including, but not limited to, monoterpene units. Thus, triterpenes of this invention also include the aglycones formed by hydrolysis of sugar units and further includes other modification of the triterpenoid compounds, whereby the modifications do not destroy the biological activity of the compounds.

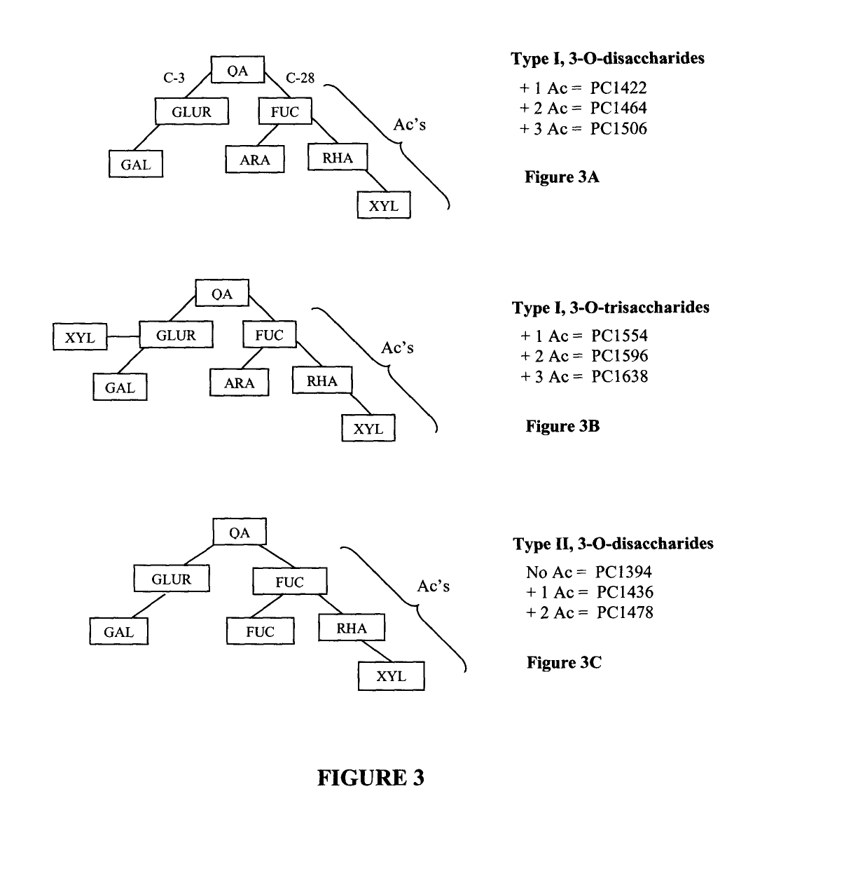

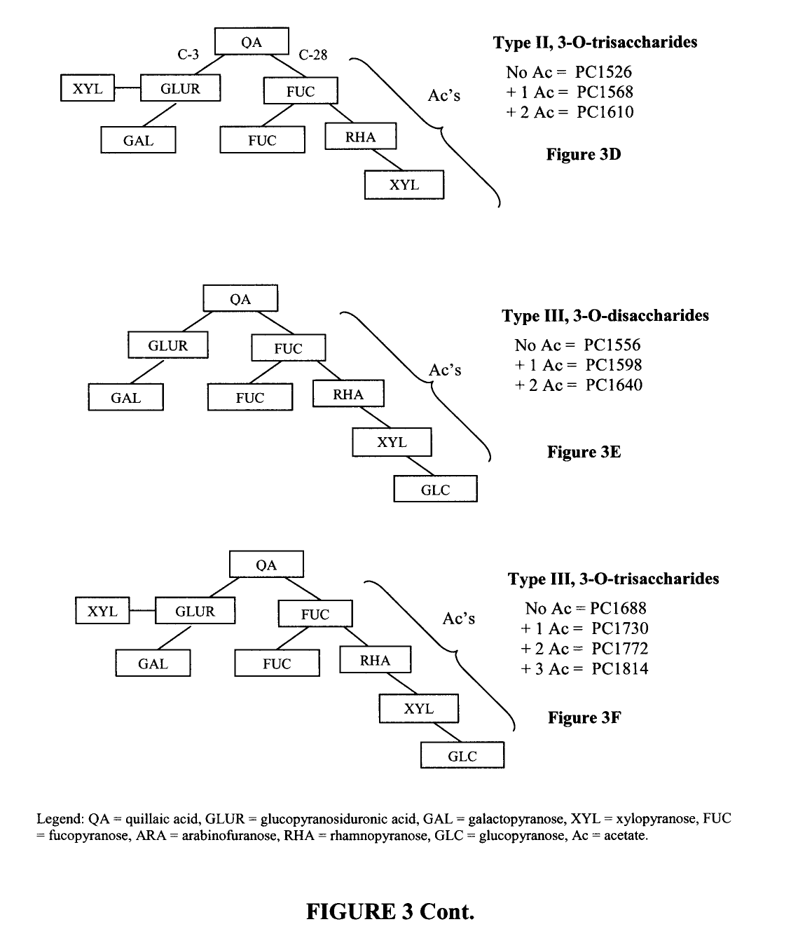

The triterpene saponin compound from the seed of Saponaria vaccaria may comprise one or more than one bisdesmosidic saponin. The bisdesmosidic saponin comprises sugar groups that are acylated, for example the saponin may comprise 1, 2, 3 or more acyl groups (see FIG. 3). The bisdesmosidic saponin may have a molecular weight of 1448, 1464, 1422, 1506, 1526, 1596, 1688 or those listed in Table 1 (see Example 2). The present invention provides a Saponaria vaccaria bisdesmosidicbisdesmosidic saponin of molecular weight 1448. The present invention also provides a Saponaria vaccaria bisdesmosidic saponin of molecular weight 1464. The present invention also provides a Saponaria vaccaria bisdesmosidic saponin of molecular weight 1422. The present invention provides a Saponaria vaccaria bisdesmosidic saponin of molecular weight 1506. The present invention also provides a Saponaria vaccaria bisdesmosidic saponin of molecular weight 1526. The present invention also provides a Saponaria vaccaria bisdesmosidic saponin of molecular weight 1596. The present invention also provides two bisdesmosidic saponins of molecular weight 1688, referred to as 1688 isomer 1 (1688-1), and 1688 isomer 2 (1688-2).

The bisdesmosidic saponin of molecular weight 1448, 1464, 1422, 1506, 1526 , 1596, 1688-1 or 1688-2 may have been separated and purified from a Saponaria vaccaria tissue, such as the seed of Saponaria vaccaria, however a person of skill in the art will recognize that a chemically synthesized compound having the structure of the Saponaria vaccaria bisdesmosidic saponins of the present invention may be made using standard techniques known in the art and the present invention is therefore not limited to the naturally isolated compound.

As shown herein in the Examples, compositions comprising bisdesmosidic saponin of molecular weight 1448, 1464, 1422, 1506, 1526-1596, 1688-1 and 1688-2 isolated from Saponaria vaccaria seed or root stimulated apoptosis in human prostate cancer (PC-3) cells, human breast cancer (MDA-MB-231 and MCF-7) cells, and human colon cancer cells (HT-29 and WiDr). Combinations of the saponins described herein may also be used for inducing apoptosis, treating cancer cells, or both.

Apoptosis is a normal physiologic process of programmed cell death, which occurs during embryonic development and during maintenance of tissue homeostasis. The process of apoptosis can be subdivided into a series of metabolic changes in apoptotic cells. Individual enzymatic steps of several regulatory or signal transduction pathways can be assayed to demonstrate that apoptosis is occurring in a cell or cell population, or that the process of cell death is disrupted. Apoptotic cells show characteristic morphological features including: cell shrinkage and rounding, condensation of the cytoplasm and nucleus, chromatin aggregation, membrane blebbing and the formation of apoptotic bodies.

Techniques to assay enzymatic and signaling processes involved in apoptosis have been developed as standard protocols. One example of an early step in apoptosis, is the release of cytochrome C from mitochondria and the activation of caspase-3. Induction of the caspases (a series of cytosolic proteases) is one of the most consistently observed features of apoptosis. Caspases are a family of proteolytic enzymes that transmit the apoptotic signal using a proteolytic cascade. They are synthesized as inactive pro-caspases, which are activated by proteolytic cleavage, and can then cleave and activate other caspases and downstream targets. Caspases 3 and 7 are effector caspases, central to the caspase cascade pathways. In particular, caspase-3 plays a central role in the process. When caspases are activated, they cleave target proteins; one of the most important of these is poly-(ADP-ribose) polymerase (PARP), which is a protein located in the nucleus. Therefore, assays detecting release of cytochrome C, detecting caspase-3 activity and detecting PARP degradation are effective determinants of apoptosis. The loss of mitochondrial membrane potential is known to occur during apoptosis. To analyze mitochondrial membrane potential, a cationic dye may be used which accumulates in the mitochondria of healthy cells. Collapse of the mitochondrial membrane potential during apoptosis prevents the dye from accumulating in the mitochondria resulting in a decrease in fluorescent intensity that can be detected with flow cytometry. Dual Sensor: MitoCasp™ (Cell Technology) used herein in the Examples is an assay that measures caspase 3/7 activity and cells with decreased mitochondrial membrane potential. In this assay, caspase 3/7 activity is detected using a cell permeable carboxyfluorescein labeled caspase inhibitor, which binds covalently to active caspases 3 and 7. Cells that contain bound inhibitor are detected by an increase in fluorescence when samples are analyzed by flow cytometry.

Another apoptotic assay is the Annexin-V detection of extracellular phosphatidlyserine (PS). Normally, phosphatidylserine is localized on the inner membrane of the plasmalemma. However, during the early stages of apoptosis, externalization of PS takes place. Annexin-V is a calcium binding protein which binds to PS and can be observed with annexin-V-FITC staining by flow cytometry (Martin, et al., 1995). Alexa Fluor® 488-labeled Annexin V, which has a high affinity for PS, is used to detect apoptotic cells by binding to PS on the outer surface of the cell. Propidium iodide staining is used concurrently to detect dead cells. When cells are analyzed by flow cytometry apoptotic cells show Annexin V staining, late apoptotic cells show PI and Annexin V staining, and dead cells show only PI staining. The Vybrant® Apoptosis Assay Kit #2 (Molecular Probes™) used herein in the Examples is an assay that measures Annexin-V positive cells.

In a still further apoptotic assay, apoptosis can be measured by the APO LOGIX™ Carboxyfluorescein Caspase 9 Detection Kit. The APO LOGIX™ Carboxyfluorescein Caspase 9 Detection Kit uses a cell permeable carboxyfluorescein labeled caspase inhibitor, which binds covalently to active caspases 9. Caspase 9 is involved in initiating apoptosis through the mitochondrial pathway (Budihardjo I., et al. 1999). Cells that contain bound inhibitor are detected by an increase in fluorescence when samples are analyzed by flow cytometry.

Apoptosis can also be measured by Hoechst 33342 Staining. Cells undergoing apoptosis show several characteristic morphological changes. These changes include cell shrinkage and rounding, and the formation of cytoplasmic blebs on the cell surface. Nuclear material condenses along the edge of the nucleus followed by complete condensation and nuclear fragmentation (Hacker, G., 2000). Apoptotic cells eventually break up into membrane bound vesicles, which are known as apoptotic bodies. Hoechst 33342 is a fluorescent DNA-binding dye that allows visualization of chromatin distribution within a cell. Apoptotic nuclei have highly condensed chromatin, often in crescent shapes around the periphery of the nucleus.

As herein described in the Examples, PC-3, MDA-MB-231, HT-29 and WiDr cells were treated with the isolated bisdesmosidic saponin for various different time periods and at different concentrations and compared to untreated cells using three standard assays known in the art, Dual Sensor: MitoCasp™ (Cell Technology), Vybrant® Apoptosis Assay Kit #2 (Molecular Probes™) and APO LOGIX™ Carboxyfluorescein Caspase 9 Detection Kit. The isolated bisdesmosidic saponins of molecular weight 1448, 1464, 1422, 1526, 1596 and 1688 (isomers 1 and 2) exhibit apoptosis in the human cancer cell lines at concentrations where there is little or no cytotoxicity to normal human cells. In non-cancerous cell lines (fibroblast cell line CRL-2522) treated with the isolated bisdesmosidic saponins of molecular weight 1448, 1464, 1422, 1526 and 1596, no or little apoptosis was seen.

Accordingly, the present invention provides a method of stimulating apoptosis in cancer cells, comprising treating the cancer cells with the saponin extract of the present invention. Preferably, the bisdesmosidic saponin comprises sugar groups that are acylated, for example the saponin may comprise 1, 2, 3 or more acyl groups. More particularly, the present invention provides a method of stimulating apoptosis in cancer cells, comprising treating the cancer cells with a saponin extract from Saponaria vaccaria comprising one or more than one bisdesmosidic saponin of molecular weight 1448, 1464, 1422, 1506, 1526, 1596, 1688-1 or 1688-2. In a further embodiment the present invention provides a method of stimulating apoptosis in cancer cells, comprising treating the cancer cells with a Saponaria vaccaria bisdesmosidic saponin having a molecular weight selected from the group consisting of molecular weight 1448, 1464, 1422, 1526, 1596 and 1688 (isomer 1 or isomer 2). The bisdesmosidic saponin may have molecular weight 1448. The bisdesmosidic saponin may have molecular weight 1464. The bisdesmosidic saponin may have molecular weight 1422. The bisdesmosidic saponin may have molecular weight 1526. The bisdesmosidic saponin may have molecular weight 1596. The bisdesmosidic saponin may have molecular weight 1688 (isomer 1 or isomer 2). The cancer cells may be human prostate cancer cells, such as but not limited to PC-3 cells, or human breast cancer cells, such as but not limited to MDA-MB-231 cells or human colon cancer cells, such as but not limited to HT-29 and WiDr cells.

Abnormal proliferation is defined as uncontrolled cell growth that occurs in mammalian cells in the pathological state known as cancer. This process eventually results in the loss of control of apoptosis in cancer cells. This can occur in steps, generally referred to as: 1) initiation, which is defined as the stage when an external agent or stimulus triggers a genetic change in one or more cells; and 2) promotion, which is defined as the stage involving further genetic and metabolic changes, which can include inflammation. During the “promotion stage”, cells begin a metabolic transition to a stage of cellular growth in which apoptosis is blocked. Tumour formation and progression is a multistage process involving a series of genetic changes.

Malignant cells are defined as cancer cells that escape normal growth control mechanisms through a series of genetic alterations resulting in metabolic changes. These changes can be inherited or acquired, and result from mutations, chromosomal aberrations, and epigenetic changes leading to activation of protoncogenes, and inactivation of tumor-suppressor genes. Genes involved in signal transduction pathways that control cell cycle regulation, promote differentiation, sense DNA damage and initiate repair mechanisms and regulate apoptosis are frequently modified in cancers. Cells have multiple parallel regulatory mechanisms, thus several genetic modifications are required to cause a cell to become malignant.

As bisdesmosidic saponin of molecular weight 1448, 1464, 1422, 1526, 1596, and 1688 (isomer 1 or isomer 2) isolated from Saponaria vaccaria seed stimulated apoptosis in human prostate cancer (PC-3) cells, human breast cancer (MDA-MB-231 and MCF-7) cells and human colon cancer cells (HT-29 and WiDr), but did not stimulate apoptosis in non-cancer (CRL-2522) cells, the bisdesmosidic saponins may be useful for the treatment of cancer. Using mixtures of triterpene saponins as disclosed herein for stimulating apoptosis is also contemplated.

Accordingly, the present invention provides a pharmaceutical composition comprising a therapeutically effective amount of the saponin extract of the present invention. More particularly, the present invention provides a pharmaceutical composition comprising a therapeutically effective amount of a saponin extract from Saponaria vaccaria comprising one or more than one bisdesmosidic saponin of molecular weight 1448, 1464, 1422, 1526 1596, or 1688 (isomer 1, isomer 2) and a pharmaceutically acceptable carrier. In an alternative embodiment the present invention provides a pharmaceutical composition comprising a therapeutically effective amount of a Saponaria vaccaria bisdesmosidic saponin having a molecular weight selected from the group consisting of molecular weight 1448, 1464, 1422, 1526, 1596, 1688 (isomer 1, isomer 2) and a combination thereof, and a pharmaceutically acceptable carrier. The bisdesmosidic saponin may have molecular weight 1448. The bisdesmosidic saponin may have molecular weight 1464. The bisdesmosidic saponin may have molecular weight 1422. The bisdesmosidic saponin may have molecular weight 1526. The bisdesmosidic saponin may have molecular weight 1596. The bisdesmosidic saponin may have -molecular weight 1688 (isomer 1, isomer 2).

A “therapeutically effective amount” refers to the amount of an agent sufficient to induce a desired biological result, i.e., treatment of cancer. That result may be alleviation of the signs, symptoms, or causes of a disease, or any other desired alteration of a biological system. The amount that is “effective” may vary from subject to subject, and can be readily determined by one of skill in the art. The compound may be administered to a subject in need thereof using any desired delivery system, for example, injection, skin patch or orally as can be readily determined by one of skill in the art. Preferred modes of administration are parenteral routes, including subcutaneous, intradermal, intravenous, intramuscular, or intraperitoneal injection, or infusion techniques.

By “pharmaceutically acceptable carrier” is meant a compound that is not biologically or otherwise undesirable, i.e., the carrier may be administered to a patient, mammal or other animal without causing any undesirable biological effects or interacting in a deleterious manner.

The present invention also provides a method of treating a subject with cancer comprising administering the pharmaceutical composition of the present invention to the subject. The cancer may be prostate cancer, breast cancer or colon cancer or other cancers as would be apparent to one of skill in the art.

A method for inducing apoptosis in a malignant mammalian cell in a mammal comprising administering to the mammal a therapeutically effective amount of the pharmaceutical compositions described herein is also provided. The malignant cell may be a skin cell, a colon cell, a uterine cell, an ovarian cell, a pancreatic cell, a lung cell, a bladder cell, a prostate cell, a renal cell, a breast cell, or a combination thereof.

In a further aspect, the present invention provides use of the pharmaceutical composition of the present invention for treating cancer. More particularly, the present invention provides use of a saponin extract comprising one or more than one Saponaria vaccaria bisdesmosidic saponin of molecular weight 1448, 1464, 1422, 1526, 1596 or 1688 (isomer 1, isomer 2) for treating cancer. Furthermore, the present invention provides use of a Saponaria vaccaria bisdesmosidic saponin of molecular weight 1448, 1464, 1422, 1526, 1596 or 1688 (isomer 1, isomer 2) and a combination thereof, for treating cancer. The cancer may be prostrate cancer, breast₌cancer or colon cancer. The bisdesmosidic saponin may have molecular weight 1448. The bisdesmosidic saponin may have molecular weight 1464. The bisdesmosidic saponin may have molecular weight 1422. The bisdesmosidic saponin may have molecular weight 1526. The bisdesmosidic saponin may have molecular weight 1596. The bisdesmosidic saponin may have molecular weight 1688 (isomer 1, isomer 2).

Separation and purification techniques are well known in the art and the fractionation of plant extracts and the recovery of terpenoid saponins by various chromographic techniques are generally well established. It is noted that utility of the saponin compounds of the present invention does not necessarily require that these compounds are always isolated and used or presented in a purified state. A crude saponin extract containing a mixture of one or more bisdesmosidic saponins of the present invention may be used instead of the purified compound. Additionally, partial purification using fewer steps or alternative methods may be acceptable for specific uses.

Although many strategies are possible for preparation of crude saponin mixtures, methods typically include extraction with methanol, ethanol, water or aqueous alcohol mixtures. Frequently a defatting step using petroleum ether or equivalent solvent or a dialysis step to remove small water-soluble molecules may be conducted prior to saponin extraction. Extraction of Saponaria seed derived materials may be with aqueous methanol solutions.

The analysis of triterpene saponins by Thin-Layer Chromatography (TLC) is highly useful for many aspects of saponin research and development. The TLC method is versatile and easily applied without specialized equipment. The applications and results achieved for TLC analysis of saponins is presented in detail in U.S. Pat. No. 7,105,186 (which is incorporated herein by reference). Additionally, the use of silica gel column chromatography and polymeric resins such as Sephadex or Amberchrom (polystyrene resin), the use of medium pressure liquid chromatography (MPLC) and high pressure liquid chromatography (HPLC) alone or in combination with mass spectrophotometry are widely described in the art as recited by U.S. Pat. No. 7,105,186.

Any single method of chromatographic procedure may be insufficient alone to isolate a pure saponin, generally a combination of classical chromatographic techniques and modern high-resolution methods such as HPLC and Counter Current Chromatography will be required for the separation of many of the individual saponins from a complex mixture.

The present invention provides a method of preparing an isolated saponin extract from Saponaria vaccaria with apoptosis stimulating activity comprising: a) milling seed of Saponaria vaccaria; and b) treating the milled seed with a solvent to produce the saponin extract. The solvent may comprises methanol, for example but not limited to 50% to 100% methanol, or any amount therebetween, for example 50, 55, 60, 65, 70, 75, 80, 85, 90, 95, 100% methanol or any amount there between, for example 60% or 70% methanol. The volume of the solvent may reduced by evaporation to dryness. The saponin extract may comprise on a dry weight basis, approximately 25 to about 30% saponins, 8% cyclopeptides, 6% phenolics, and 45% saccharides.

An isolated bisdesmosidic saponin composition comprising at least about 40% by weight bisdesmosidic saponins may be prepared from the saponin extract by titrating the saponin extract with 70% to 100% methanol or any amount there between, for example 100% methanol, allowing undissolved materials to settle, filtering to recover a solid residue, and reducing to dryness in vacuo.

The present invention further provides a method of preparing an isolated bisdesmosidic saponin composition from Saponaria vaccaria comprising at least 60% by weight bisdesmosidic saponins (preferably at least 70% by weight bisdesmosidic saponins) with apoptosis stimulating activity, the method comprising: a) milling seed of Saponaria vaccaria; b) treating the milled seed with a solvent to produce a saponin mixture; c) cooling the saponin mixture to precipitate bisdesmosidic saponins; and d) recovering the precipitate comprising the isolated bisdesmosidic saponin composition.

The isolated bisdesmosidic saponin composition from Saponaria vaccaria produced by the method of the present invention may comprise between 60-90% by weight bisdesmosidic saponin, or any amount therebetween, such 60, 62, 64, 66, 68, 70, 72, 74, 76, 78, 80, 82, 84, 86, 88, or 90%, or any amount therebetween.

The saponin mixture is cooled to a temperature where the saponins precipitate. Precipitation of a bisdesmosidic saponin rich composition by cooling has the advantage of reduced cost compared to known extraction and purification methods using chromatographic procedures that require expensive resins.

The solvent may be aqueous methanol, such as but not limited to 70% aqueous methanol. The saponin mixture may be cooled to a temperature of about minus 10 to about minus 30 degrees C., or any temperature therebetween, such as minus 10, 12, 14, 16, 18, 20, 22, 24, 26, 28, or 30 degrees C., for about 36 to about 60 hours or any amount of time therebetween, such as 36, 37, 38, 39, 40, 41, 42, 43, 44, 45, 46, 47, 48, 49, 50, 51, 52, 53, 54, 55, 56, 57, 58, 59 or 60 hours or any time therebetween. The saponin mixture may be cooled for longer than 60 hours, however this may be unlikely to increase precipitation. The step of recovering the precipitate (step d) may be carried out by decantation and reduction to dryness in vacuo to produce a solid residue comprising the bisdesmosidic saponin composition. The method may further comprise addition of a second solvent to the saponin mixture following step (b). The second solvent may be ethanol. The amount of ethanol added to the saponin mixture, may be sufficient to produce an extract comprising 40-50% by volume ethanol, or any amount therebetween, such as 40, 41, 42, 43, 44, 45, 46, 47, 48, 49, or 50% by volume ethanol.

The isolated saponins of the present invention may be 75-98% pure, or any purity therebetween, such as 75, 76, 77, 78, 79, 80, 81, 82, 83, 84, 85, 86, 87, 88, 89, 90, 91, 92, 93, 94, 95, 96, 97 or 98% pure or any purity therebetween. Alternatively, saponins characterized having increased purity may be obtained by performing multiple rounds of chromatography, in addition different solvent and pH systems may be used with different rounds of chromatography.

Saponins, in particular 3-O-trisachharide bisdesmosidics, for example those having molecular weight 1596 (PC1596) or 1772 (PC1772) may be isolated from the roots of Saponaria vaccaria plants, using methods described herein, or as are known in the art. Furthermore, bisdesmosidic saponin having molecular weight 1506 and 1464 may be converted to a bisdesmosidic saponin having molecular weight 1422 by basic treatment using methods that are known to one of skill in the art.

As described herein in the Examples, Saponaria vaccaria bisdesmosidic saponin compounds of different molecular weight were isolated from a bisdesmosidic saponin rich extract by column chromatography using a linear gradient of methanol/water from for example 60%-100% methanol. It is demonstrated in the Examples that bisdesmosidic saponin compounds of molecular weight 1448, 1464, 1422, 1526 1596, and 1688 (isomers 1 and 2) stimulated apoptosis in human prostate cancer (PC-3) cells, human breast cancer (MDA-MB-231 and MCF-7) cells and human colon cancer (HT-29 and WiDr) cells.

Accordingly, the present invention provides a method of preparing an isolated saponin extract from Saponaria vaccaria comprising bisdesmosidic saponin of molecular weight 1448, the method comprising: a) milling seed of Saponaria vaccaria; b) treating the milled seed with a solvent thereby extracting a saponin composition; c) applying the saponin composition to a column to effect separation of individual saponins; and d) recovering the bisdesmosidic saponin of molecular weight 1448. The bisdesmosidic saponin of molecular weight 1448 may be recovered in step d) by elution of the column with 100% methanol.

The present invention also provides a method of preparing an isolated saponin extract from Saponaria vaccaria comprising bisdesmosidic saponin of molecular weight 1464, the method comprising: a) milling seed of Saponaria vaccaria; b) treating the milled seed with a solvent thereby extracting a saponin composition; c) applying the saponin composition to a column to effect separation of individual saponins; and d) recovering the bisdesmosidic saponin of molecular weight 1464. The bisdesmosidic saponin of molecular weight 1464 may be recovered in step d) by elution of the column with 90% methanol.

The present invention also provides a method of preparing an isolated saponin extract from Saponaria vaccaria comprising bisdesmosidic saponin of molecular weight 1596, the method comprising: a) drying and pulverizing roots from Saponaria vaccaria; b) treating the pulverized roots with a solvent thereby extracting a saponin composition; c) applying the saponin composition to a column to effect separation of individual saponins; and d) recovering the bisdesmosidic saponin of molecular weight 1596. The bisdesmosidic saponin of molecular weight 1596 may be recovered in step d) by elution of the column with 80% methanol.

The present invention also provides a method of preparing an isolated saponin extract from Saponaria vaccaria comprising bisdesmosidic saponin of molecular weight 1526, the method comprising: a) milling seed of Saponaria vaccaria; b) treating the milled seed with a solvent thereby extracting a saponin composition; c) applying the saponin composition to a column to effect separation of individual saponins; and d) recovering the bisdesmosidic saponin of molecular weight 1526. The bisdesmosidic saponin of molecular weight 1526 may be recovered in step d) by elution of the column with at least 70-75% methanol.

The present invention also provides a method of preparing an isolated saponin extract from Saponaria vaccaria comprising bisdesmosidic saponin of molecular weight 1422, the method comprising: a) milling seed of Saponaria vaccaria; b) treating the milled seed with a solvent thereby extracting a saponin composition; c) applying the saponin composition to a column to effect separation of individual saponins; d) recovering bisdesmosidic saponin of molecular weight 1464; e) dissolving the bisdesmosidic saponin of molecular weight 1464 in a solvent and treating with a base to obtain a basic composition; f) applying the basic composition to a column to effect separation of individual saponins; and g) recovering the bisdesmosidic saponin of molecular weight 1422. The bisdesmosidic saponin of molecular weight 1464 may be recovered in step d) by elution of the column with at least 80% methanol. The bisdesmosidic saponin of molecular weight 1422 may be recovered in step g) by elution of the column with 75% methanol. The solvent in step e) may be methanol, such as 30% methanol. The base in step e) may be an hydroxide such as ammonium hydroxide.

The present invention also provides a method of preparing an isolated saponin extract from Saponaria vaccaria comprising bisdesmosidic saponin of molecular weight 1688 (isomer 1), the method comprising: a) drying and pulverizing roots from Saponaria vaccaria; b) treating the pulverized roots with a solvent thereby extracting a saponin composition; c) applying the saponin composition to a column to effect separation of individual saponins; and d) recovering the bisdesmosidic saponin of molecular weight 1772. The bisdesmosidic saponin of molecular weight 1772 is e) dissolved in a solvent and treated with a base to affect deacetylation and f) applied to a column to affect separation of saponins. The bisdesmosidic saponin 1772 may be recovered in step d) from the column typically by elution with 80-85% methanol. The bisdesmosidic saponin molecular weight 1688 (isomer 1) may be recovered in step f) by elution with 65% methanol. The base in step e) may be an hydroxide such as ammonium hydroxide.

The present invention provides a method of preparing an isolated saponin extract from Saponaria vaccaria comprising bisdesmosidic saponins of molecular weight 1688 (isomers 1 and 2), the method comprising: a) milling seed of Saponaria vaccaria; b) treating the milled seed with a solvent thereby extracting a saponin composition; c) dissolving said saponin mixture in a solvent and treating the solution with base to affect deacetylation; and d) separating individual saponins on a column to afford 1688-1 and 1688-2. The bisdesmosidic saponins may be recovered in step d) typically by elution with 65-70% methanol.

The present invention will be further illustrated in the following examples.

### EXAMPLES

### Example 1

### Method for Isolating a Crude Saponin Mixture from Saponaria vaccaria Seed

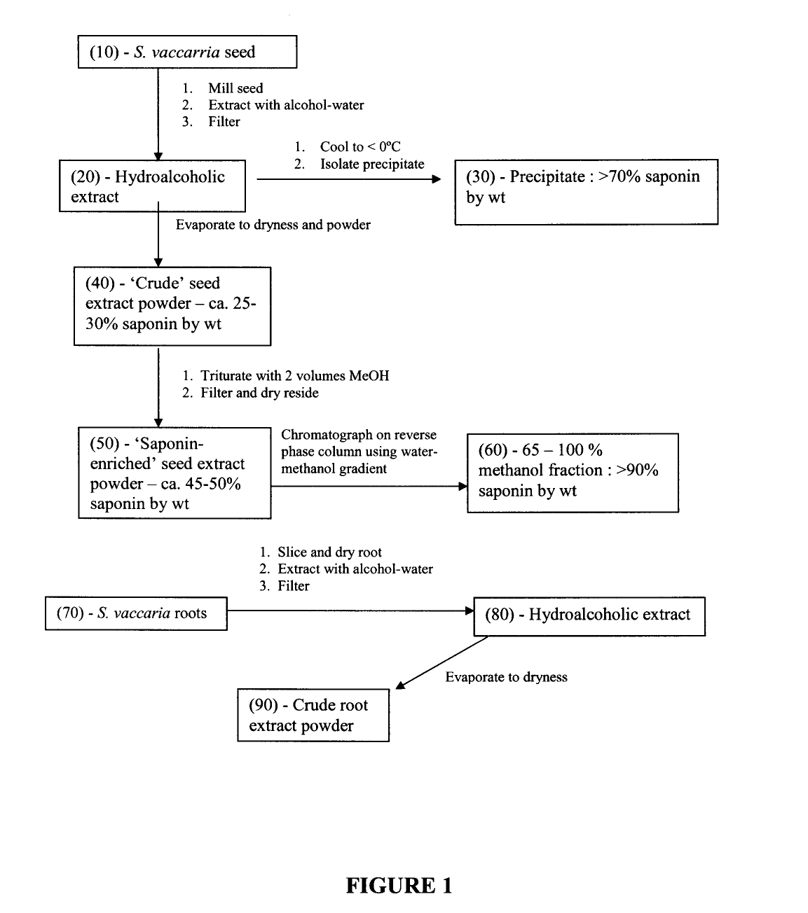

The process for preparation of a crude saponin mixture from Saponaria vaccaria seed or root is shown schematically in FIG. 1. Bulk Saponaria vaccaria seed harvested mechanically from field grown plants was cleaned by screening and air classification to remove debris and any foreign seeds. Five hundred grams of cleaned seed was ground to a fine powder in a mill (coffee grinder, seed mill or Waring blender, etc). A de-fatted ground seed was produced by washing the ground seed with ethyl acetate (or equivalent such as hexane) to remove lipids and then allowed to air dry. The de-fatted ground seed was mixed with 1200 ml 60% methanol containing 100 mg citric acid and allowed to stand for one day at room temperature. The resultant mixture was filtered through a sintered glass filter with minimal or no vacuum. The filter cake was re-extracted with 1000 ml 60% methanol (other concentration of methanol such as 70% can be used) resulting in a hydroalcoholic extract of approximately 2 litres. A crude saponin mixture was obtained by placing the hydroalcoholic extract in a tray in a fume hood and allowing the filtrate to evaporate to produce a dry seed extract. The aqueous extract concentrate may be adjusted to pH 5.0 if required by addition of citric acid. The seed extract concentrate was diluted with 500 ml ethanol and 100 ml butanol and evaporated on a rotary evaporator until the foam point is reached. The process is repeated until the water is removed and a solid residue remains. The last traces of water were removed under high vacuum. The resultant solid is powdered and stored in glass bottles and is termed the “dry seed extract”. The powder contains approximately 28% saponins. Additional components include: free sugars, phenolics and cyclopeptides. A further concentration of the dry seed extract to approximately 45-50% saponins was prepared by mixing the 28% saponin containing powder in two volumes of methanol. The mixture was allowed to settle and was filtered. The filtrate comprises largely non-polar cyclopeptides and the majority of the monodesmoside saponins. The solid residue was dried under vacuum and powdered. The resultant powder was approximately 45-50% bisdesmosidic saponins with phenolics and other components.

A preparation comprising largely monodesmosidic saponins and cyclopeptides was prepared by adjusting the pH of the de-fatted aqueous concentrate from Example 1 to pH 7.5 with sodium bicarbonate and extracting 3 times with 125 ml n-butanol. The combined butanol washing was concentrated by evaporation.

Alternatively, a preparation comprised predominantly of bisdesmosidic saponins (>70%) was prepared by treatment of 800 ml of the hydroalcoholic extract with 500 ml ethanol and placement in a freezer at −20 C for several days. A precipitate of >70% bisdesmosidic saponins was recovered by decanting off the liquid.

### Example 2

### Characterization of Individual Saponins from Saponaria vaccaria var. “Scott”

The chemical profile of saponins present in Saponaria vaccaria seed was determined by high performance liquid chromatographic methods using photodiode array and single quadrupole electrospray mass detection (HPLC-MS-PAD) for analysis and profiling of bisdesmosidic saponins. A summary of these results is presented in Table 1.

### HPLC-MS-PAD Analysis

A Waters Alliance 2695 chromatography system with inline degasses, coupled to a ZQ 2000 mass detector and a 2996 PAD was used for analyses. Waters MassLynx v 4.0 software was used for data acquisition and manipulation. The columns used were Waters Symmetry RP C₁₈ (150 H 2.1 mm i.d.; 3.5 μ), Waters Sunfire RP C₁₈ (150 H 2.1 mm i.d.; 3.5 μ), or Phenomenex (Torrance, Calif., USA) Synergi MAX-RP 80A C₁₂ (250 H 2.0 mm i.d.; 4 μ). The flow rate with the Waters columns was 0.2 mL/min, and with the Phenomenex column 0.15 mL/min. Columns were maintained at 35° C. during runs. The binary solvent systems used were:
solvent A, 0.12% acetic acid in 10% acetonitrile (aq, v/v); and
solvent B 0.12% acetic acid in 100% acetonitrile.

Gradients used were:
0-3 min, 75% A/25% B; 3-25 min 75% A/25% B to 50% A/50% B; 25-28 min, 25% A/75% B to 100% B; 28-33 min, 100% B; and
0-8 min, 90% A/10% B; 8-31 min, 90% A/10% B to 50% A/50% B; 31-33 min 50% A/50% B to 100% B; 33-48 min 100% B.

Injection volumes of 5 μL were typical.

Unless otherwise noted, the mass detector parameters (ESE) were set to: capillary (kV) 2.70; cone (V) −30→−90.0 over a mass range of 400-1900; extractor (V) −3.50, RF Lens (V) −0.7. PAD was performed over the range 200-400 nm, and saponins were monitored at 209 nm.

### Extraction and Fractionation

For isolation of wild type saponins: Wild type seed (10 g) was ground and de-fatted with hexane. The de-fatted meal was extracted with 70% methanol (60 mL) by stirring at ambient temperature for 20 h. The meal was separated by centrifugation and extracted a second time with 70% methanol (25 mL) for 4 hr. The combined methanolic extract was concentrated in vacuo to afford an amber solid (0.7 g), which was dissolved in water and applied to a conditioned and equilibrated 10 mL SPE cartridge and eluted sequentially with 20-40 mL portions of water, 30% -60% methanol, and 70-100% methanol. Saponins were obtained in the 70 100% fractions which were combined and concentrated in vacuo to afford a white powder (230 mg).

### Saponification, Isolation and Comparison of Prosapogenins

S. vaccaria saponins (100-200 mg) were dissolved in 1 M sodium hydroxide (5 mL) and stirred under a nitrogen atmosphere for 3 days at ambient temperature or heated at 80° C. for 4 hr. The solution was carefully neutralized with 1 M hydrochloric acid, acidified with a small amount of citric acid (ca. 50 mg) and applied to a 5 mL SPE cartridge and sequentially eluted with water, 30%, 70%, and 100% methanol. The prosapogenins were obtained in the 70-100% fractions. Prosapogenins from samples were run on Phenomenex and Sunfire columns using both gradients, as outlined above.

TABLE 1
Bisdesmosidic saponins observed in Saponaria vaccaria “Scott”

| | | | | | Rₜ |
|---|---|---|---|---|---|
| [M − | | | | | (min) |
| H]⁻ | | 3-O- | Com- | Fragment | (single |
| m/z | Aglycone | substituents | pound | ions, m/z | run) |
| 1394 | Quillaic acid | Disaccharide | Unknown | 823, 485 | 10.48 |
| 1406 | Gypsogenin | Disaccharide | Vaccaro- | 807, 469 | 17.81 |
| | | | side G | | |
| 1422 | Quillaic acid | Disaccharide | Vaccaro- | 823, 485 | 13.55 |
| | | | side E | | |
| 1436 | Quillaic acid | Disaccharide | Unknown | 823, 485 | 18.36 |
| 1448 | Gypsogenin | Disaccharide | Segetoside | 807, 469 | 22.07 |
| | | | H | | |
| 1464 | Quillaic acid | Disaccharide | Segetoside | 823, 485 | 18.36 |
| | | | I | | |
| 1478 | Quillaic acid | Disaccharide | Unknown | 823, 485 | 23.59 |
| 1506 | Quillaic acid | Disaccharide | Segetoside | 823, 485 | 20.87 |
| | | | I Ac | | |
| 1526 | Quillaic acid | Trisaccharide | Unknown | 955, 485 | 11.27 |
| 1538 | Gypsogenin | Trisaccharide | New | 939, 469 | 17.58 |
| | | | saponin | | |
| 1554 | Quillaic acid | Trisaccharide | New | 955, 485 | 13.52 |
| | | | saponin | | |
| 1556 | Quillaic acid | Disaccharide | Unknown | 823, 485 | 10.60 |
| 1568 | Quillaic acid | Trisaccharide | Unknown | 955, 485 | 18.23 |
| 1580 | Gypsogenin | Trisaccharide | New | 939, 469 | 21.69 |
| | | | saponin | | |
| 1596 | Quillaic acid | Trisaccharide | New | 955, 485 | 18.13 |
| | | | saponin | | |
| 1598 | Quillaic acid | Disaccharide | Unknown | 823, 485 | 17.91 |
| 1610 | Quillaic acid | Trisaccharide | Unknown | 955, 485 | 23.27 |
| 1626 | Quillaic acid | Trisaccharide^{a} | New | 985, 485 | 17.51 |
| | | | saponin | | |
| 1638 | Quillaic acid | Trisaccharide | New | 955, 485 | 20.52 |
| | | | saponin | | |
| 1640 | Quillaic acid | Disaccharide | Unknown | 823, 485 | 23.06 |
| 1688-1 | Quillaic acid | Trisaccharide | Unknown | 955, 485 | 9.18 |
| 1688-2 | Quillaic acid | Trisaccharide | Unknown | 955, 485 | 10.69 |
| 1730 | Quillaic acid | Trisaccharide | Unknown | 955, 485 | 11.91 |
| 1730 | Quillaic acid | Trisaccharide | Unknown | 955, 485 | 17.76 |
| 1772 | Quillaic acid | Trisaccharide | Unknown | 955, 485 | 22.75 |
| 1814 | Quillaic acid | Trisaccharide | Unknown | 955, 485 | 23.53 |

^{a}Trisaccharide = glucuronic acid, galactose, hexose (unknown)

Retention times were obtained by selected ion extraction from total ion current (TIC) of appropriate quasi-molecular ion. Aglycone type (sapogenin) was determined from combined extracted mass spectrum obtained at a cone voltage of 90 V. Slight differences in retention time on the chromatograms are due to differences in tubing lengths leading to detectors. Disaccharide=3-O-βD-Galp-(1→2)-β-D-GlcpA; Trisaccharide=3-O-β-D-Xylp-(1→3)-βD-Galp-(1→2)]β-D-GlcpA.

### Example 3

### Purification of Individual Saponin Species

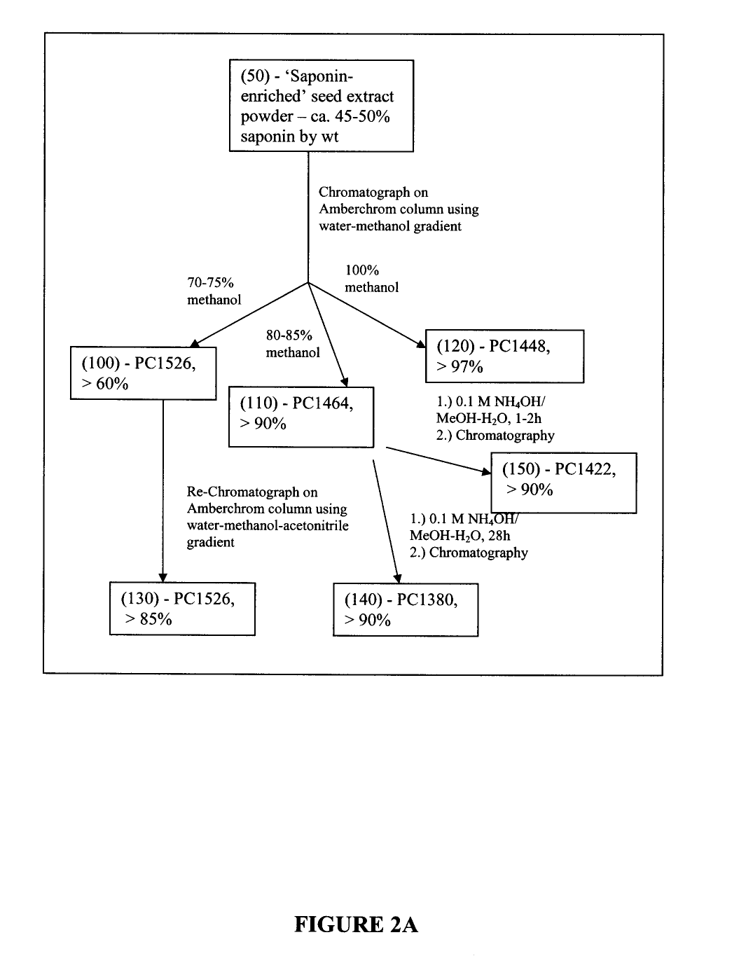

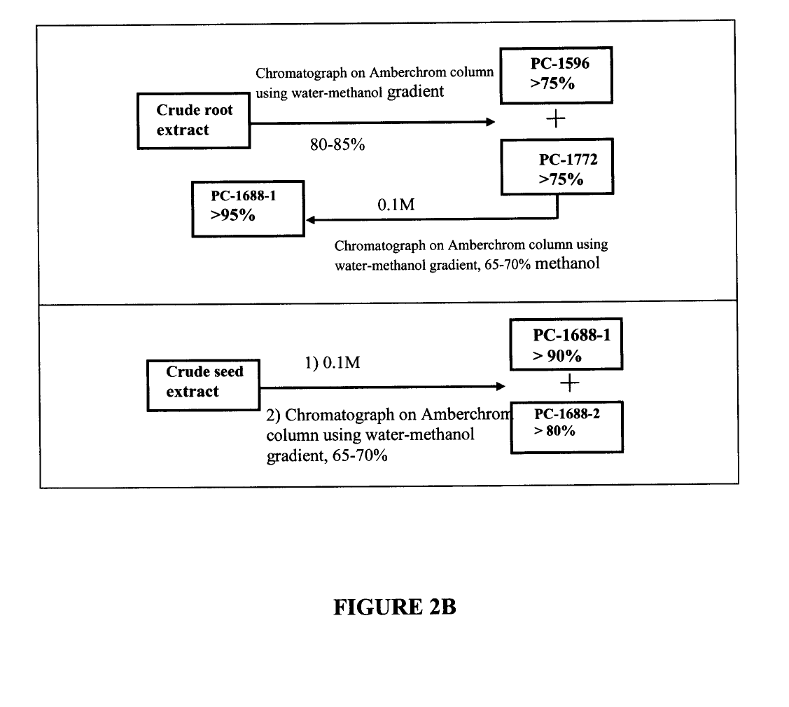

A schematic representation of the method of separating and purifying of saponins PC1526, 1464, 1448, 1422, 1380, 1596, 1688-1 and 1688-2 present in seeds and roots of S. vaccaria is shown in FIGS. 2A and 2B.

For isolation of PC1526, 1464, and 1448, a hydroalcoholic extract was prepared from seed of a saponaria variety having a low titer of 3-O-trisaccharide type saponins. A mixture of bisdesmosidic saponins (45-50% powder), freed from non-polar cyclic peptides by trituration of crude extract powder with methanol (i.e. saponin-enriched seed extract, FIG. 1), was chromatographed on an Amberchrom 300M reverse phase resin.

A solution estimated to contain 25 μm of mainly bisdesmosidic saponins (low in 3-O-trisaccharide types) in approximately 1 L 20% methanol was applied to a packed column containing 1.5 L of Amberchrom 300M resin in water. After application of the saponin solution, the column was eluted with 1 L of 20% methanol containing 0.01% acetic acid and 1 L of 50% methanol. Thereafter, a linear gradient of methanol/water from 60-100% methanol was applied with 90 fractions of ca. 100 ml volume being collected over the gradient range. PC1464 was observed in several fractions eluted with a gradient of ≧80% methanol. Evaporation of the best fraction afforded 960 mg PC1464 in >90% purity, as a white amorphous powder. PC-1448 was observed in fractions eluted with 100% methanol. Concentration of fractions containing PC1448 followed by decolorization with charcoal and removal of solvent, led to 240 mg of >97% PC1448 (by HPLC), as a white amorphous powder. PC1526 enriched solutions were observed in fractions eluted with 70-75% methanol. Evaporation of fractions containing approximately 60% (HPLC) or greater PC1526 afforded 700 mg material which was re-chromatographed on Amberchrom 300S resin (ca. 250 ml) using a ternary solvent system consisting of 50% methanol-water plus acetonitrile (1:1 methanol-water plus acetonitrile from 5-40% of total volume). Collection of 50 ml fractions led to isolation of several fractions (20-25% acetonitrile) containing PC1526 in >85% purity (HPLC). Evaporation of combined fractions afforded 182 mg.

For isolation of PC1596, mature roots of Saponaria were collected, sliced, and air dried. Dried roots were pulverized and extracted with hot (typically 50° C.) aqueous methanol (typically 70%). After 2-3 h the mixture was filtered and solvents removed to afford an amorphous saponin root powder. The powder was dissolve in a minimum amount of 30% methanol and chromatographed as above. In this manner PC1596 of >75% purity was obtained in fractions eluted with 80% methanol.

PC1422 was most conveniently prepared from PC1464 (140 mg) by dissolving PC1464 in 30% methanol (aq, 20 ml) and adding conc. ammonium hydroxide to a final concentration of 0.1 M NH4OH (ca. 0.15 ml). After 2 h at ambient temp, the solution was neutralized with citric acid, and applied to an Amberchrom resin column in water, and eluted with a water-methanol gradient as described above to afford PC1422 (78 mg, >90%) mainly in fractions eluted with 75% methanol.

PC1380 was prepared from PC1464 as for PC1422 except the solution was stirred at ambient temperature for 28 hours, and PC1380 was eluted with 55-60% methanol.

PC1688-1 was prepared by drying and pulverizing roots from Saponaria vaccaria; treating the pulverized roots with a solvent thereby extracting a saponin composition; applying the saponin composition to a column containing Amberchrom and using a water-methaol gradient to effect separation of individual saponins; and recovering the bisdesmosidic saponin of molecular weight 1772.

The bisdesmosidic saponin of molecular weight 1772 is dissolved in a solvent and treated with a base to affect deacetylation and applied to a column containing Amberchrom to affect separation of saponins. The bisdesmosidic saponin 1772 may be recovered from the column typically by elution with 80-85% methanol. The bisdesmosidic saponin molecular weight 1688 (isomer 1) may be recovered typically by elution with 65% methanol. The base may be an hydroxide such as ammonium hydroxide.

PC1688 (isomers 1 and 2) are prepared by milling seed of Saponaria vaccaria; treating the milled seed with a solvent thereby extracting a saponin composition; dissolving the saponin mixture in a solvent and treating the solution with base to affect deacetylation; and separating individual saponins on a column containing Amberchrom to afford 1688-1 and 1688-2. The bisdesmosidic saponins in may be recovered by elution with 65-70% methanol.

Saponins from Calendula officinalis were extracted by using standard extraction methods for Calendula saponins know in the art.

The nomenclature adopted for the saponins is based on molecular weight determined by mass spectrometry, rather than names such as segetoside H and segetoside I that are used in the literature. The preface “PC” stands for Prairie Carnation as a distinction from cow cockle and wild type Saponarias.

FIG. 3 shows a tentative family tree of different saponins of the Quillaic Acid type found in Saponaria vaccaria (Prairie Carnation). QA represents Quilliac Acid the terpeniod backbone (alglycone). Sugars are attached at two locations. The sugar groups that are attached can be acylated or not.

### Example 4

### Treatment of Prostate Cancer (PC-3) Cells with PC1448

PC-3 cells (ATCC #CRL-1435) were derived from a prostate adenocarcinoma (Kaighn et al., 1979). They were maintained in RPMI 1640 medium (Gibco) supplemented with 10% fetal bovine serum (Cansera), 100 U/mL penicillin, and 100 μg/mL streptomycin and 0.25 μg/mL amphotericin B. Cultures were maintained in medium at 37° C. with 5% CO₂ and 100% relative humidity.

To harvest adherent cells, the existing medium was removed and cells were dissociated with 0.25% (w/v) trypsin-EDTA·4Na in Hank's Balanced Salt Solution (Gibco). To block the proteolytic action of the trypsin, the cells were resuspended in an excess of media containing fetal bovine serum. Cells were transferred to a 15 mL conical centrifuge tube and pelleted by centrifugation at 500×g for 5 min. Cell pellets were resuspended in media, and an aliquot of the cells were seeded into a new flask.

Three assays were used to determine if PC1448 could induce apoptosis in PC-3 cells:
1. Dual Sensor: MitoCasp™ (Cell Technology);
2. Vybrant® Apoptosis Assay Kit #2 (Molecular Probes™); and
3. APO LOGIX™ Carboxyfluorescein Caspase 9 Detection Kit (Cell Technology).

PC-3 cells were seeded in tissue culture flasks with regular growth medium and maintained under normal conditions. After cells had adhered, the tissue culture medium was replaced with medium containing PC1448 dissolved in DMSO with the final concentration of DMSO not exceeding 0.05%. For each assay, a flask of untreated cells was grown in media containing the same amount of DMSO as in the PC1448 treated samples. Following incubation under normal growth conditions for the specified time, cells were washed once with cold phosphate-buffered saline (PBS) (137 mM NaCl, 2.7 mM KCl, 4.3 mM Na₂HPO₄.7H₂O, 1.4 mM KH₂PO₄) and harvested as previously described. Assays were carried out according to instructions given in kits. All Flow Cytometry was performed using a Coulter Epics XL (Beckman).

### Dual Sensor: MitoCasp™

Following PC1448 treatment, adherent and non-adherent cells were harvested and washed with cold PBS. Following centrifugation at 500×g for 5 min, cell pellets were resuspended in PBS at 3.3×10⁶ cells/mL. 300 μL cell suspension was mixed with 10 μL each of 30X Mitochondria Membrane Potential Dye and 30X Caspase 3/7 detection reagent. Following incubation for 60 min under normal growth conditions, cells were washed twice with 1× wash buffer and resuspended in 0.5 mL 1× wash buffer. Samples were analyzed by flow cytometry measuring the fluorescence emissions from 515 nm to 530 nm for caspase detection, and 574 nm to 600 nm for mitochondrial membrane potential detection.

A time course study from 0 to 20 hr was conducted with PC-3 cells treated with 14 μM PC1448 and analyzed using the Dual Sensor:MitoCasp™. Results that demonstrate a significant increase in the cells with caspase 3/7 activity and concomitantly a dramatic increase in cells with decreased mitochondrial membrane potential are shown in Table 2.

TABLE 2
Apoptosis measured in PC-3 cells treated with 14 μM PC1448 for 20
hr measured using the Dual Sensor: MitoCasp ™ Assay

| | Cells with caspase | Cells with decreased mitochondrial |
|---|---|---|
| 14 μM PC1448 | 3/7 activity (%) | membrane potential (%) |
| Untreated | 24.6 | 4.5 |
| 05 hr | 13.0 | 38.0 |
| 10 hr | 29.2 | 72.5 |
| 15 hr | 42.9 | 89.4 |
| 20 hr | 57.2 | 94.1 |

After only five hours of exposure to PC1448 there was a rapid increase in the numbers of cells with decreased mitochondrial membrane potential. Further exposure times showed the vast majority of cells with low mitochondrial membrane potential and Capsase 3/7 activity indicating onset of apoptosis.

In comparison to untreated PC-3 cells, the PC1448 treated PC-3 cells showed a progressive time dependent increase representing approximately a doubling of the cells with measurable caspase 3/7 activity over the 20 hr exposure period. Additionally, there was about a 20 fold increase (4.5% to 94.1%) in cells with a decreased mitochondrial membrane potential following treatment with PC1448. A highly significant impact was observed after just five hours exposure.

A similar set of experiments was conducted to test a range of concentrations of PC1448 (from 7-17 μM). Following treatment of PC-3 cells with PC1448 for 26 hours, a dose dependant increase in apoptotic cells was observed as shown in Table 3.

TABLE 3
Apoptosis in PC-3 cells treated with different concentrations of PC1448
for 26 hr measured using the Dual Sensor: MitoCasp ™ Assay

| PC1448 | | |
|---|---|---|
| concentration | Cells with caspase | Cells with decreased mitochondrial |
| (μM) | 3/7 activity (%) | membrane potential (%) |
| Untreated | 10.1 | 2.8 |
| 7 | 77.8 | 24.8 |
| 10 | 86.2 | 30.8 |
| 14 | 84.0 | 52.0 |
| 17 | 77.7 | 41.9 |

The largest increase in caspase 3/7 activity was with cells treated with 10 μM PC1448, however all treated cells showed a dramatic increase (a 9 fold increase) in caspase 3/7 activity, even at 7 μM, the lowest concentration tested. The greatest change in cells with decreased mitochondrial membrane potential occurred with 14 μM PC1448, however, all treated cells showed large increases even at the lowest concentration tested.

After exposure of PC-3 cells to PC1448 at 7, 10, 14, 17₁1M concentrations for 26 hours, there was a dramatic increase in the number of cells displaying low mitochondrial membrane potential and increased levels of Caspase 3/7, indicating the induction of apoptosis.

### Vybrant® Apoptosis Assay Kit #2

A second time course study was conducted to measure apoptosis in PC-3 cells treated with 14 μM PC1448 for different times using the Vybrant® Apoptosis Assay Kit #2. The results are given in Table 4 and indicate a three fold increase in Annexin V positive cells, when compared to untreated PC-3 cells, following a 24 hr treatment with PC1448.

TABLE 4
Apoptosis in PC-3 cells treated with 14 μM PC1448 for various
times measured using the Vybrant ® Apoptosis Assay Kit #2

| 14 μM PC1448 | Annexin-V positive cells (%) |
|---|---|
| Untreated | 11.3 |
| 06 hr | 12.4 |
| 16 hr | 28.3 |
| 24 hr | 34.7 |

### APO LOGIX™ Carboxyfluorescein Caspase 9 Detection Kit

Further evidence that PC1448 stimulates apoptosis was evident from detection of caspase 9 activity using the APO LOGIX™ Carboxyfluorescein Caspase 9 Detection Kit. Following treatment of PC-3 cells with 14 μM PC1448 for different times, the APO LOGIX™ Carboxyfluorescein Caspase 9 Detection Kit was used to measure apoptosis. The results are given in Table 5, and indicate that PC1448 treatment caused an increase in PC-3 cells with caspase 9 activity. The largest increase was seen at 20 hr with a six fold increase in PC-3 cells showing caspase 9 activity compared with untreated cells.

TABLE 5
Apoptosis in PC-3 cells treated with 14 μM PC1448 over time measured
using the APO LOGIX ™ Carboxyfluorescein Caspase 9 Detection Kit

| 14 μM PC1448 | Caspase 9 positive cells (%) |
|---|---|
| Untreated | 8.5 |
| 10 hr | 37.3 |
| 15 hr | 44.2 |
| 20 hr | 48.0 |
| 40 hr | 29.4 |

A concentration range experiment was conducted with PC-3 cells treated with PC1448 for 20 hr at concentrations ranging from 7-17 μM using APO LOGIX™ Carboxyfluorescein Caspase 9 Detection Kit. The results are given in Table 6, which demonstrates that the number of cells with caspase 9 activity increased with PC1448 concentration up to 14 μM, at which concentration the cells showed a six fold increase compared to untreated PC-3 cells. All treated cells regardless of PC1448 concentration showed large increases in Caspase 9 activity.

TABLE 6
Apoptosis in PC-3 cells treated with different concentrations of
PC1448 for 20 hr measured using the APO LOGIX ™ Carboxyfluorescein
Caspase 9 Detection Kit

| PC1448 concentration | |
|---|---|
| (μM) | Caspase 9 positive cells (%) |
| Untreated | 13.2 |
| 7 | 47.7 |
| 10 | 73.9 |
| 14 | 75.3 |
| 17 | 65.6 |

### Example 5

### Treatment of Breast Cancer (MDA-MB-231) Cells with PC1448

The MDA-MB-231 cell line (ATCC #HTB-26) was derived from the pleural effusion of a patient with a breast adenocarcinoma (Cailleau et al., 1974). The cells were maintained in RPMI 1640 (Gibco) supplemented with 10% fetal bovine serum (Camera), 100 U/mL penicillin, 100 μg/mL streptomycin and 0.25 μg/mL amphotericin B. Cultures were maintained in medium at 37° C. with 5% CO₂ and 100% relative humidity.

MDA-MB-231 cells were treated with PC1448 and assayed for apoptosis as previously described for PC-3 cells.

### Vybrant® Apoptosis Assay Kit #2

Following treatment with 14 μM PC1448 for various different times, MDA-MB-231 cells were assessed for apoptitic activity using the Vybrant® Apoptosis Assay Kit #2 . Results are shown in Table 7 and indicate a 2.7 fold increase in Annexin V positive treated cells, compared to untreated cells, after only 12 hours exposure to PC1448.

TABLE 7
Apoptosis in MDA-MB-231 cells treated with 14 μM PC1448 for
different times measured using the Vybrant ® Apoptosis Assay
Kit #2

| 14 μM PC1448 | Annexin-V positive cells (%) |
|---|---|
| Untreated | 17.1 |
| 12 hr | 46.3 |
| 24 hr | 37.9 |
| 48 hr | 37.9 |

### APO LOGIX™ Carboxyfluorescein Caspase 9 Detection Kit

Additionally, MDA-MB-231 cells treated with 14 μM PC1448 for various different times were analyzed for apoptotic activity using the APO LOGIX™ Carboxyfluorescein Caspase 9 Detection Kit. Results are shown in Table 8 and show an increased number of cells treated with PC1448 with Caspase 9 activity compared to untreated cells, in as little as 10 hours. The maximum effect was a 2.5 fold increase in cells with caspase 9 activity after 40 hours exposure to PC1448. These results show the rapid rise in Caspase 9 activity after cells are exposed to PC1448 indicating cells are under going apoptosis.

TABLE 8
Apoptosis in MDA-MB-231 cells treated with 14 μM PC1448 for
different times measured using the APO LOGIX ™ Carboxyfluorescein
Caspase 9 Detection Kit

| 14 μM PC1448 | Caspase 9 positive cells (%) |
|---|---|
| Untreated | 15.2 |
| 10 hr | 23.2 |
| 15 hr | 29.8 |
| 20 hr | 30.5 |
| 40 hr | 39.4 |

### Example 6

### Treatment of Fibroblast (CRL-2552) Cells with PC1448

CRL-2522 cells, acquired from ATCC, (American Type Culture Collection) are normal fibroblast cells that originated from human foreskin. CRL-2522 cells were maintained in RPMI 1640 medium (Gibco) supplemented with 10% fetal bovine serum (Cansera), 100 U/mL penicillin, 100 μg/mL streptomycin and 0.25 μg/mL amphotericin B. Cultures were maintained in medium at 37° C. with 5% CO₂ and 100% relative humidity.

CRL-2522 cells were treated with PC1448 and assayed for apoptosis as described above for PC-3 cells.

### Dual Sensor:MitoCasp™ Assay

CRL-2522 cells were treated with 14 μM PC1448 for various different times and analyzed using the Dual Sensor:MitoCasp™ Assay. Results are shown in Table 9 and show that exposure of CRL-2522 cells to PC1448, even over prolonged times, caused no increase in cells with lowered mitochondrial membrane potential. Additionally, there was no evidence of the increase in Caspase 3/7 activity until an extended culture time (30 hours). These observations support the view that PC1448 at 14 μM concentration had no significant effect on apoptosis in normal fibroblast cells.

TABLE 9
Apoptosis in CRL-2522 cells treated with 14 μM PC1448 for different
times measured using the Dual Sensor: MitoCasp ™ Assay

| 14 μM | Cells with caspase 3/7 | Cells with decreased mitochondrial |
|---|---|---|
| PC1448 | activity (%) | membrane potential (%) |
| Untreated | 3.1 | 1.9 |
| 10 hr | 3.7 | 1.4 |
| 20 hr | 5.5 | 1.4 |
| 30 hr | 32.8 | 0.8 |

### Vybrant® Apoptosis Assay

CRL-2522 cells were treated with 14 μM PC1448 for various different times, and the Vybrant® Apoptosis Assay Kit #2 was used to measure apoptosis. The results are shown in Table 10. No meaningful changes in the number of CRL-2522 cells showing Annexin V staining following PC1448 treatment were evident.

TABLE 10
Apoptosis in CRL-2522 cells treated with 14 μM PC1448 for different
times measured using the Vybrant ® Apoptosis Assay Kit #2

| 14 μM PC1448 | Annexin-V positive cells (%) |
|---|---|
| Untreated | 13.5 |
| 10 hr | 17.2 |
| 20 hr | 9.3 |
| 40 hr | 18.2 |

### APO LOGIX™ Carboxyfluorescein Caspase 9 Detection Kit

A still further indication that PC1448 does not stimulate apoptosis in normal fibroblast CRL-2522 cells was obtained using the APO LOGIX™ Carboxyfluorescein Caspase 9 Detection Kit. Results from treatment of CRL-2522 cells with 14 μM PC1448 for various different times are shown in Table 11. No increase in Caspase 9 positive cells was observed following a 10 to 48 hr treatment with PC1448.

TABLE 11
Apoptosis in CRL-2522 cells treated with 14 μM PC1448 for
different times measured using the APO LOGIX ™
Carboxyfluorescein Caspase 9 Detection Kit

| 14 μM PC1448 | Caspase 9 positive cells (%) |
|---|---|
| Untreated | 8.9 |
| 10 hr | 15.9 |
| 24 hr | 10.6 |
| 48 hr | 9.5 |

### Example 7

### Treatment of Prostate Cancer (PC-3) Cells with PC1464

The ability of PC1464, a second saponin purified from S. vaccaria in Example 3, to induce apoptosis in PC-3 cells was tested using the apoptosis assays described above. PC-3 cells were treated with PC1464 using the methods described for PC1448.

### Dual Sensor: MitoCasp™ Assay

PC-3 cells treated with 7 μM PC1464 for various different times were analyzed using the Dual Sensor: MitoCasp™ Assay. Results are shown in Table 12. Exposure to PC1464 at a concentration of 7 μM induced apoptosis in PC-3 cells in a time dependent fashion. Increases in apoptotic activity were apparent after only 10 hours and increased dramatically thereafter. After exposure of PC-3 cells to 7 μM PC1464 for 30 hours, the number of cells with caspase 3/7 activity increased six fold whereas cells with decreased mitochondrial membrane potential increased more than ten fold.

TABLE 12
Apoptosis in PC-3 cells treated with 7 μM PC1464 for different times
measured using the Dual Sensor: MitoCasp ™ Assay

| 7 μM | Cells with caspase 3/7 | Cells with decreased mitochondrial |
|---|---|---|
| PC1464 | activity (%) | membrane potential (%) |
| Untreated | 9.0 | 1.8 |
| 10 hr | 12.2 | 5.1 |
| 20 hr | 40.1 | 21.4 |
| 30 hr | 54.6 | 51.2 |

The strong induction of apoptosis by PC1464 at a concentration of 7 μM suggested that lower concentrations may also be effective. A series of decreasing concentrations of PC1464 (1.75-7 μM) were tested for 26 hour using the Dual Sensor: MitoCasp™ Assay. The results are shown in Table 13, which shows clearly the induction of apoptosis at all concentrations tested but with significant effect at concentrations of 3.5 μM or higher.

TABLE 13
Apoptosis in PC-3 cells treated with different concentrations of PC1464
for 26 hr measured using the Dual Sensor: MitoCasp ™ Assay

| | | Cells with |
|---|---|---|
| PC1464 concentration | Cells with caspase 3/7 | decreased mitochondrial |
| μM | activity (%) | membrane potential (%) |
| Untreated | 7.8 | 1.7 |
| 1.75 | 8.5 | 3.2 |
| 3.50 | 40.4 | 14.8 |
| 5.25 | 49.5 | 20.3 |
| 7.00 | 59.4 | 24.1 |

### Vybrant® Apoptosis Assay Kit #2

The Vybrant® Apoptosis Assay Kit #2 was used to measure apoptosis in PC-3 cells treated with 7 μM or 14 μM PC1464 for various different times as shown in Table 14 and Table 15.

TABLE 14
Apoptosis measured in PC-3 cells treated with 7 μM PC1464 for different
times measured using the Vybrant ® Apoptosis Assay Kit #2

| 7 μM PC1464 | Annexin-V positive cells (%) |
|---|---|
| Untreated | 25.4 |
| 10 hr | 21.0 |
| 20 hr | 30.9 |
| 30 hr | 44.7 |

TABLE 15
Apoptosis in PC-3 cells treated with 14 μM PC1464 for different
times measured using the Vybrant ® Apoptosis Assay Kit #2

| 14 μM PC1464 | Annexin-V positive cells (%) |
|---|---|
| Untreated | 8.5 |
| 10 hr | 11.5 |
| 20 hr | 28.9 |
| 30 hr | 49.4 |

The results indicate that PC-3 cells showed a three to six fold increase in Annexin V positive cells following a 30 hr treatment with PC1464, compared to untreated cells.

### Example 8

### Treatment of Breast Cancer (MDA-MB-231) Cells with PC1464

MDA-MB-231 breast cancer cells were treated with PC1464 as previously described for PC1448. Apoptosis assays were performed as described above.

### Dual Sensor: MitoCasp™ Assay

Following treatment of MDA-MB-231 cells with 3.5 μM PC1464 for various different times, the Dual Sensor: MitoCasp™ Assay was used to measure apoptosis. The results are given in Table 16. PC1464 was able to induce apoptosis in MDA-MB-231 cells at a concentration of 3.5 μM in 12 hours. After exposure of MDA-MB-231 cells to 3.5 μM PC1464 for 36 hours, the number of cells with caspase 3/7 activity increased by two fold, whereas the number of cells with reduced mitochondrial membrane potential increased eight fold.

TABLE 16
Apoptosis is measured in MDA-MB-231 cells treated with 3.5 μM PC1464
for different times using the Dual Sensor: MitoCasp ™ Assay

| 3.5 μM | Cells with caspase 3/7 | Cells with decreased mitochondrial |
|---|---|---|
| PC1464 | activity (%) | membrane potential (%) |
| Untreated | 15.0 | 6.6 |
| 12 hr | 20.0 | 19.9 |
| 24 hr | 27.5 | 51.5 |
| 36 hr | 29.1 | 52.0 |

### Vybrant® Apoptosis Assay Kit #2

MDA-MB-231 cells were treated with 7 μM PC1464 for various different times using the Vybrant® Apoptosis Assay Kit #2. Results are shown in Table 17. After a 36 hour exposure to PC1464, there was a 3 fold increase in Annexin V positive MDA-MB-231 cells compared to untreated MDA-MB-231 cells.

TABLE 17
Apoptosis in MDA-MB-231 cells treated with 7 μM PC1464 for different
times measured using the Vybrant ® Apoptosis Assay Kit #2

| 7 μM PC1464 | Annexin-V positive cells (%) |
|---|---|
| Untreated | 17.2 |
| 12 hr | 28.8 |
| 24 hr | 39.7 |
| 36 hr | 49.3 |

### Example 9

### Treatment of Fibroblast (CRL-2552) Cells with PC1464

Normal non-cancerous CRL-2522 fibroblast cells were treated with PC1464 as previously described for PC1448. Apoptosis assays were carried out as described above.

### Dual Sensor: MitoCasp™ Assay

The Dual Sensor: MitoCasp™ Assay was used to analyze CRL-2522 cells treated with 7 μM PC1464 for various different times. The results are shown in Table 18 and demonstrate that PC1464 had a minimal effect on CRL-2522 cells. There was a modest increase in CRL-2522 treated cells with Caspase 3/7 activity compared to untreated CRL-2522 cells, however such increases are expected after extended periods of culture. Essentially no changes in the numbers of treated cells with decreased mitochondrial membrane potential were detected.

TABLE 18
Apoptosis in CRL-2522 fibroblast cells treated with 7 μM PC1464 for
different times measured using the Dual Sensor: MitoCasp ™ Assay

| 7 μM | Cells with caspase 3/7 | Cells with decreased mitochondrial |
|---|---|---|
| PC1464 | activity (%) | membrane potential (%) |
| Untreated | 2.7 | 3.9 |
| 10 hr | 3.2 | 4.4 |
| 20 hr | 11.7 | 4.4 |
| 30 hr | 16.9 | 5.1 |

### Vybrant® Apoptosis Assay Kit #2

CRL-2522 cells were treated with 7 μM PC1464 for various different times and the results are shown in Table 19. The results show that PC1464 had little effect on CRL-2522 cells. The percent of Annexin V positive cells increased only modestly after a 30 hour exposure.

TABLE 19
Apoptosis in CRL-2522 fibroblast cells treated with 7 μM PC1464 for
different times measured using the Vybrant ® Apoptosis Assay Kit #2

| 7 μM PC1464 | Annexin-V positive cells (%) |
|---|---|
| Untreated | 6.9 |
| 10 hr | 6.0 |
| 20 hr | 8.4 |
| 30 hr | 10.2 |

### Example 10

### Hoechst 33342 Staining

A further indication of apoptotic activity, in addition to the assays described above, can be derived from observations of changes in cell architecture evident after induction of apoptosis. Hoechst 33342 is a fluorescent DNA-binding dye that allows visualization of chromatin distribution within a cell.

Cells undergoing apoptosis show several characteristic morphological changes. These changes include cell shrinkage and rounding, and the formation of cytoplasmic blebs on the cell surface. Nuclear material condenses along the edge of the nucleus followed by complete condensation and nuclear fragmentation (Hacker, G., 2000). Apoptotic cells eventually break up into membrane bound vesicles, which are known as apoptotic bodies.

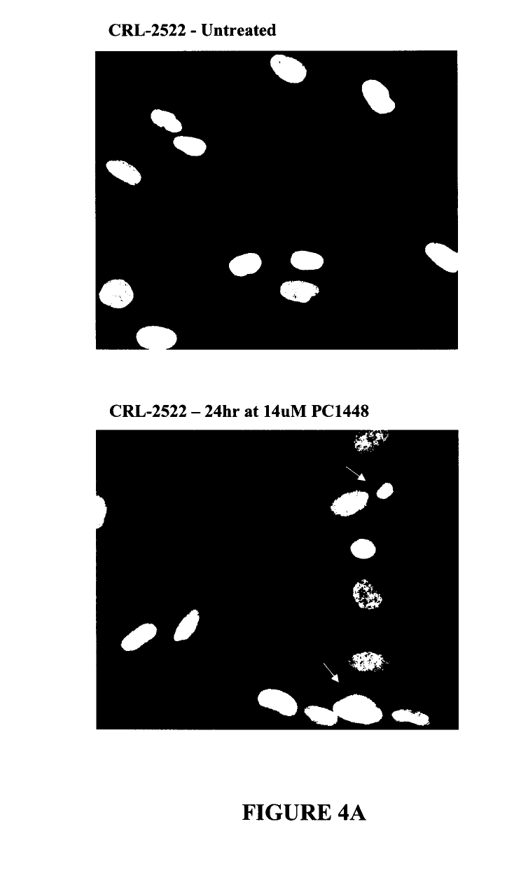

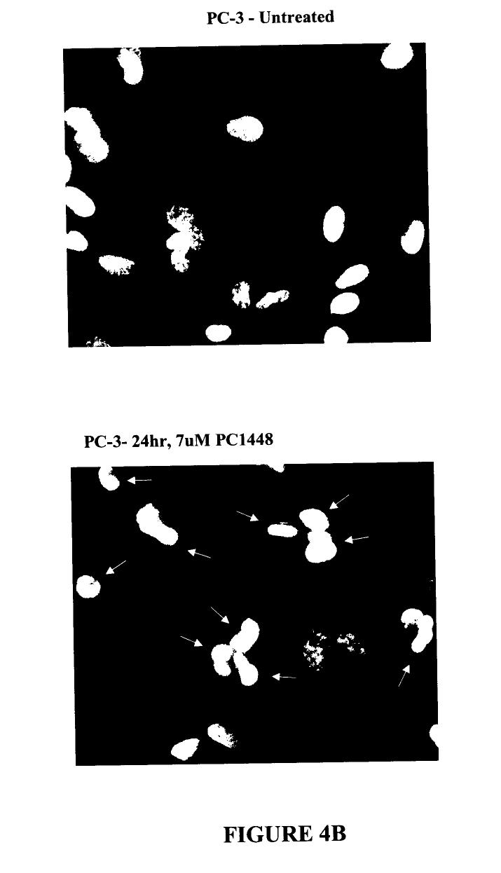

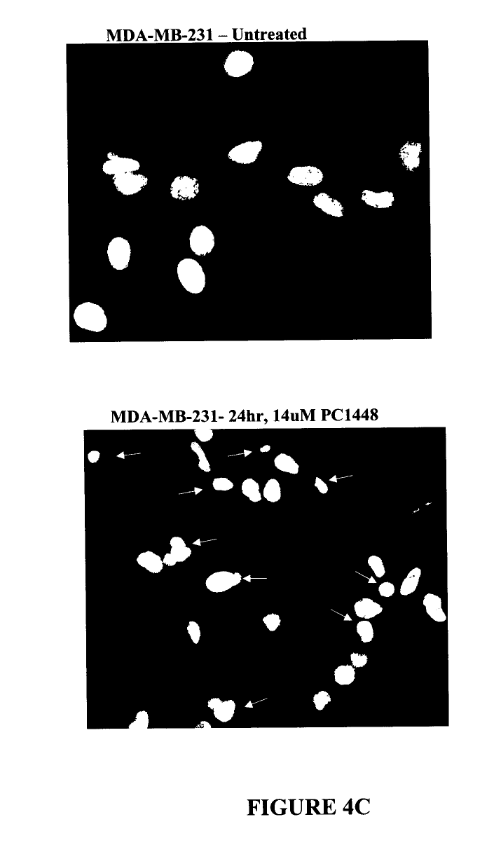

PC-3 (prostate cancer) cells, MDA-MB-231 (breast cancer) cells, and CRL-2522 (normal fibroblast) cells, were treated for 24 hours with 7 μM PC1448 and then stained with Hoechst 33342. The results are shown in FIG. 4a-4c, which indicates nuclear changes characteristic of apoptotic cells. Apoptotic cells can be identified by nuclei having highly condensed chromatin, often in crescent shapes around the periphery of the nucleus.

### Example 11

### Treatment of Prostate Cancer (PC-3) Cells with PC1422

The ability of PC1422, another saponin purified from S. vaccaria in Example 3, to induce apoptosis in PC-3 cells was tested using the apoptosis assays described above. PC-3 cells were treated with PC1422 using the methods described for PC1448 and PC1464. As shown in FIG. 3, PC1422 is the single acyl form of PC1464.

PC-3 cells were treated with decreasing concentrations of PC1422 (15.0-2.5 μM) for 36 hours and assessed using the Dual Sensor: MitoCasp™ Assay. The results are shown in Table 20, which shows clearly the induction of apoptosis at concentrations as low as 2.5 μM with the greatest effect occurring above 10 μM.

TABLE 20
Apoptosis in PC-3 cells treated with different concentrations of PC1422
for 36 hr measured using the Dual Sensor: MitoCasp ™ Assay

| | | Cells with |
|---|---|---|
| PC1422 | Cells with caspase 3/7 | decreased mitochondrial |
| concentration (μM) | activity (%) | membrane potential (%) |
| Untreated | 20.3 | 8.0 |
| 2.5 | 42.2 | 23.6 |
| 5.0 | 49.1 | 30.0 |
| 10.0 | 55.7 | 53.7 |
| 15.0 | 57.1 | 53.3 |

### Example 12

### Treatment of Breast Cancer (MDA-MB-231) Cells with PC1422

MDA-MB-231 breast cancer cells were treated with PC1422 as previously described for PC1448 and PC1464. Apoptosis assays were performed as described above.

MDA-MB-231 cells were treated with decreasing concentrations of PC1422 (15.0-2.5 μM) for 36 hours and assessed using the Dual Sensor: MitoCasp™ Assay. The results are shown in Table 21, which shows clearly the induction of apoptosis at concentrations as low as 2.5 μM with the greatest effect occurring at 15 μM.

TABLE 21
Apoptosis in MDA-MB-231 cells treated with different concentrations of
PC1422 for 36 hr measured using the Dual Sensor: MitoCasp ™ Assay

| | | Cells with |
|---|---|---|
| PC1422 | Cells with caspase 3/7 | decreased mitochondrial |
| concentration (μM) | activity (%) | membrane potential (%) |
| Untreated | 6.4 | 3.0 |
| 2.5 | 21.1 | 13.7 |
| 5.0 | 32.2 | 32.9 |
| 10.0 | 41.6 | 46.0 |
| 15.0 | 26.7 | 65.2 |

### Example 13

### Treatment of Fibroblast (CRL-2552) Cells with PC1422

Normal non-cancerous CRL-2522 fibroblast cells were treated with PC1422 as previously described for PC1448 and PC1464. Apoptosis assays were carried out as described above.

CRL-2522 cells were treated with decreasing concentrations of PC1422 (15.0-2.5 μM) for 36 hours and assessed using the Dual Sensor: MitoCasp™ Assay. The results are shown in Table 22. There was a modest increase in CRL-2522 treated cells with Caspase 3/7 activity and decreased mitochondrial membrane potential compared to untreated CRL-2522 cells, however such increases are expected after extended periods of culture.

TABLE 22
Apoptosis in CRL-2522 cells treated with different concentrations of
PC1422 for 36 hr measured using the Dual Sensor: MitoCasp ™ Assay

| | | Cells with |
|---|---|---|
| PC1422 | Cells with caspase 3/7 | decreased mitochondrial |
| concentration (μM) | activity (%) | membrane potential (%) |
| Untreated | 1.7 | 2.1 |
| 2.5 | 3.1 | 3.4 |
| 5.0 | 15.3 | 5.1 |
| 10.0 | 12.8 | 19.2 |
| 15.0 | 7.3 | 26.2 |

### Example 14

### Treatment of Prostate Cancer (PC-3) Cells with PC1526

The ability of PC1526, another saponin purified from S. vaccaria in Example 3, to induce apoptosis in PC-3 cells was tested using the apoptosis assays described above. PC-3 cells were treated with PC1526 using the methods described for PC1448 and PC1464. The tentative structure of 1526 is shown in FIG. 3d.

PC-3 cells were treated with decreasing concentrations of PC1526 (5.0-1.25 RM) for 36 hours and assessed using the Dual Sensor: MitoCasp™ Assay. The results are shown in Table 23, which shows clearly the induction of apoptosis at concentrations as low as 2.5 μM with the greatest effect occurring at 5 μM.

TABLE 23
Apoptosis in PC-3 cells treated with different concentrations of PC1526
for 36 hr measured using the Dual Sensor: MitoCasp ™ Assay

| | | Cells with |
|---|---|---|
| PC1526 | Cells with caspase 3/7 | decreased mitochondrial |
| concentration (μM) | activity (%) | membrane potential (%) |
| Untreated | 11.1 | 4.9 |
| 1.25 | 7.9 | 8.6 |
| 2.5 | 37.3 | 13.0 |
| 5.0 | 65.7 | 54.1 |

### Example 15

### Treatment of Breast Cancer (MDA-MB-231) Cells with PC1526

MDA-MB-231 breast cancer cells were treated with PC1526 as previously described for PC1448 and PC1464. Apoptosis assays were performed as described above.

MDA-MB-231 cells were treated with decreasing concentrations of PC1526 (5.0-1.25 μM) for 36 hours and assessed using the Dual Sensor: MitoCasp™ Assay. The results are shown in Table 24, which shows clearly the induction of apoptosis at concentrations as low as 2.5 μM with the greatest effect occurring at 5 μM.

TABLE 24
Apoptosis in MDA-MB-231 cells treated with different concentrations of
PC1526 for 36 hr measured using the Dual Sensor: MitoCasp ™ Assay

| | | Cells with |
|---|---|---|
| PC1526 | Cells with caspase 3/7 | decreased mitochondrial |
| concentration (μM) | activity (%) | membrane potential (%) |
| Untreated | 11.8 | 3.7 |
| 1.25 | 16.0 | 5.7 |
| 2.5 | 43.2 | 13.0 |
| 5.0 | 63.2 | 37.4 |

### Example 16

### Treatment of Fibroblast (CRL-2552) Cells with PC1526

Normal non-cancerous CRL-2522 fibroblast cells were treated with PC1526 as previously described for PC1448 and PC1464. Apoptosis assays were carried out as described above.

CRL-2522 cells were treated with decreasing concentrations of PC1526 (5.0-1.25 μM) for 36 hours and assessed using the Dual Sensor: MitoCasp™ Assay. The results are shown in Table 25. There was a small increase in CRL-2522 treated cells with Caspase 3/7 activity and decreased mitochondrial membrane potential compared to untreated CRL-2522 cells, however such increases are expected after extended periods of culture.

TABLE 25
Apoptosis in CRL-2522 cells treated with different concentrations of
PC1526 for 36 hr measured using the Dual Sensor: MitoCasp ™ Assay

| | | Cells with |
|---|---|---|
| PC1526 | Cells with caspase 3/7 | decreased mitochondrial |
| concentration (μM) | activity (%) | membrane potential (%) |
| Untreated | 1.8 | 2.4 |
| 1.25 | 2.0 | 3.2 |
| 2.5 | 6.9 | 6.6 |
| 5.0 | 8.8 | 8.2 |

### Example 17

### Treatment of Prostate Cancer (PC-3) Cells with PC1596

The ability of PC1596, another saponin purified from S. vaccaria in Example 3, to induce apoptosis in PC-3 cells was tested using the apoptosis assays described above. PC-3 cells were treated with PC1596 using the methods described for PC1448 and PC1464. As shown in FIG. 3, PC1596 is the equivalent of PC1464, but with an additional xylose.

PC-3 cells were treated with decreasing concentrations of PC1596 (3.1-0.8 μM) for 36 hours and assessed using the Dual Sensor: MitoCasp™ Assay. The results are shown in Table 26, which shows clearly the induction of apoptosis at concentrations as low as 1.6 μM and above.

TABLE 26
Apoptosis in PC-3 cells treated with different concentrations of PC1596
for 36 hr measured using the Dual Sensor: MitoCasp ™ Assay

| | | Cells with |
|---|---|---|
| PC1596 | Cells with caspase 3/7 | decreased mitochondrial |
| concentration (μM) | activity (%) | membrane potential (%) |
| Untreated | 7.1 | 11.8 |
| 0.8 | 6.1 | 10.8 |
| 1.6 | 23.1 | 17.2 |
| 3.1 | 18.1 | 29.4 |

### Example 18

### Treatment of Breast Cancer (MDA-MB-231) Cells with PC1596

MDA-MB-231 breast cancer cells were treated with PC1596 as previously described for PC1448 and PC1464. Apoptosis assays were performed as described above.

MDA-MB-231 cells were treated with decreasing concentrations of PC1596 (3.1-0.8 μM) for 36 hours and assessed using the Dual Sensor: MitoCasp™ Assay. The results are shown in Table 27, which shows clearly the induction of apoptosis at concentrations as low as 1.6 μM with the greatest effect occurring at 3.1 μM.

TABLE 27
Apoptosis in MDA-MB-231 cells treated with different concentrations of
PC1596 for 36 hr measured using the Dual Sensor: MitoCasp ™ Assay

| | | Cells with |
|---|---|---|
| PC1596 | Cells with caspase 3/7 | decreased mitochondrial |
| concentration (μM) | activity (%) | membrane potential (%) |
| Untreated | 2.8 | 0.9 |
| 0.8 | 3.1 | 2.7 |
| 1.6 | 36.9 | 16.2 |
| 3.1 | 56.0 | 39.9 |

### Example 19

### Treatment of Fibroblast (CRL-2552) Cells with PC1596

Normal non-cancerous CRL-2522 fibroblast cells were treated with PC1596 as previously described for PC1448 and PC1464. Apoptosis assays were carried out as described above.

CRL-2522 cells were treated with decreasing concentrations of PC1596 (3.1-0.8 μM) for 36 hours and assessed using the Dual Sensor: MitoCasp™ Assay. The results are shown in Table 28. There was a small increase in CRL-2522 treated cells with Caspase 3/7 activity, however such increases are expected after extended periods of culture.

TABLE 28
Apoptosis in CRL-2522 cells treated with different concentrations of
PC1596 for 36 hr measured using the Dual Sensor: MitoCasp ™ Assay

| | | Cells with |
|---|---|---|
| PC1596 | Cells with caspase 3/7 | decreased mitochondrial |
| concentration (μM) | activity (%) | membrane potential (%) |
| Untreated | 2.5 | 4.2 |
| 0.8 | 3.8 | 2.9 |
| 1.6 | 3.7 | 4.7 |
| 3.1 | 14.4 | 5.6 |

### Example 20

### Treatment of Prostate Cancer (PC-3) Cells with PC1380

PC1380 is the equivalent of PC1464 without any acyl groups isolated in Example 3. PC-3 cells were treated with decreasing concentrations of PC1380 (15.0-2.5 μM) for 35 hours and assessed using the Dual Sensor: MitoCasp™ Assay. The results are shown in Table 29. There was a small increase in PC-3 treated cells with Caspase 3/7 activity and decreased mitochondrial membrane potential compared to untreated PC-3 cells.

TABLE 29
Apoptosis in PC-3 cells treated with different concentrations of PC1380
for 35 hr measured using the Dual Sensor: MitoCasp ™ Assay

| | | Cells with |
|---|---|---|
| PC1380 | Cells with caspase 3/7 | decreased mitochondrial |
| concentration (μM) | activity (%) | membrane potential (%) |
| Untreated | 11.5 | 6.3 |
| 2.5 | 11.4 | 5.8 |
| 5.0 | 15.3 | 7.1 |
| 10.0 | 14.9 | 8.7 |
| 15.0 | 31.8 | 15.9 |

### Example 21

### Treatment of Breast Cancer (MDA-MB-231) Cells with PC1380

MDA-MB-231 cells were treated with decreasing concentrations of PC1380 (15.0-2.5 μM) for 34 hours and assessed using the Dual Sensor: MitoCasp™ Assay. The results are shown in Table 30, which shows no significant apoptosis activity in MDA-MB-231 cells treated with PC1380.

TABLE 30
Apoptosis in MDA-MB-231 cells treated with different concentrations of
PC1380 for 34 hr measured using the Dual Sensor: MitoCasp ™ Assay

| | | Cells with |
|---|---|---|
| PC1380 | Cells with caspase 3/7 | decreased mitochondrial |
| concentration (μM) | activity (%) | membrane potential (%) |
| Untreated | 7.9 | 3.2 |
| 2.5 | 6.2 | 3.2 |
| 5.0 | 8.0 | 4.7 |
| 10.0 | 9.4 | 6.6 |
| 15.0 | 7.9 | 4.3 |

### Example 22

### Treatment of Fibroblast (CRL-2552) Cells with PC1380

CRL-2522 cells were treated with decreasing concentrations of PC1380 (15.0-2.5 μM) for 32 hours and assessed using the Dual Sensor: MitoCasp™ Assay. The results are shown in Table 31. As expected no significant apoptosis of CRL-2522 cells treated with PC1380 was seen.

TABLE 31
Apoptosis in CRL-2522 cells treated with different concentrations of
PC1380 for 32 hr measured using the Dual Sensor: MitoCasp ™ Assay

| | | Cells with |
|---|---|---|
| PC1380 | Cells with caspase 3/7 | decreased mitochondrial |
| concentration (μM) | activity (%) | membrane potential (%) |
| Untreated | 1.2 | 1.3 |
| 2.5 | 0.4 | 1.8 |
| 5.0 | 1.0 | 4.0 |
| 10.0 | 1.2 | 2.6 |
| 15.0 | 0.9 | 4.5 |

### Example 23

### Treatment of Prostate Cancer (PC-3) Cells with PC1448

PC-3 prostate cancer cells were treated with PC1448 as previously described. Apoptosis assays were performed as described above.

PC-3 cells were treated with decreasing concentrations of PC1448 (15.0-2.5 μM) for 37 hours and assessed using the Dual Sensor: MitoCasp™ Assay. The results are shown in Table 32, which shows clearly the induction of apoptosis at concentrations as low as 5.0 μM with the greatest effect occurring above 10 μM.

TABLE 32
Apoptosis in PC-3 cells treated with different concentrations of PC1448
for 37 hr measured using the Dual Sensor: MitoCasp ™ Assay

| | | Cells with |
|---|---|---|
| PC1448 | Cells with caspase 3/7 | decreased mitochondrial |
| concentration (μM) | activity (%) | membrane potential (%) |
| Untreated | 6.5 | 1.3 |
| 2.5 | 5.6 | 6.0 |
| 5.0 | 35.1 | 9.9 |
| 10.0 | 76.8 | 26.7 |
| 15.0 | 72.9 | 32.3 |

### Example 24

### Treatment of Breast Cancer (MDA-MB-231) Cells with PC1448

MDA-MB-231 breast cancer cells were treated with PC1448 as previously described. Apoptosis assays were performed as described above.

MDA-MB-231 cells were treated with decreasing concentrations of PC1448 (15.0-2.5 μM) for 36 hours and assessed using the Dual Sensor: MitoCasp™ Assay. The results are shown in Table 33, which shows clearly the induction of apoptosis at concentrations as low as 5.0 μM with the greatest effect occurring above 10 μM.

TABLE 33
Apoptosis in MDA-MB-231 cells treated with different concentrations of
PC1448 for 36 hr measured using the Dual Sensor: MitoCasp ™ Assay

| | | Cells with |
|---|---|---|
| PC1448 | Cells with caspase 3/7 | decreased mitochondrial |
| concentration (μM) | activity (%) | membrane potential (%) |
| Untreated | 20.8 | 9.5 |
| 2.5 | 19.3 | 9.9 |
| 5.0 | 25.8 | 12.6 |
| 10.0 | 40.0 | 17.9 |
| 15.0 | 40.9 | 20.8 |

### Example 25

### Treatment of Fibroblast (CRL-2552) cells with PC1448

Normal non-cancerous CRL-2522 fibroblast cells were treated with PC1448 as previously described. Apoptosis assays were carried out as described above.

CRL-2522 cells were treated with decreasing concentrations of PC1448 (15.0-2.5 μM) for 37 hours and assessed using the Dual Sensor: MitoCasp™ Assay. The results are shown in Table 34. There was variable increase in CRL-2522 treated cells with Caspase 3/7 activity and decreased mitochondrial membrane potential compared to untreated CRL-2522 cells, however such variable increases are expected after extended periods of culture.

TABLE 34
Apoptosis in CRL-2522 cells treated with different concentrations of
PC1448 for 37 hr measured using the Dual Sensor: MitoCasp ™ Assay

| | | Cells with |
|---|---|---|
| PC1448 | Cells with caspase 3/7 | decreased mitochondrial |
| concentration (μM) | activity (%) | membrane potential (%) |
| Untreated | 2.3 | 2.8 |
| 2.5 | 1.7 | 5.6 |
| 5.0 | 4.0 | 5.0 |
| 10.0 | 21.9 | 4.6 |
| 15.0 | 9.4 | 18.5 |

### Example 26

### Treatment of Prostate Cancer (PC-3) Cells with PC1464

PC-3 prostate cancer cells were treated with PC1464 as previously described. Apoptosis assays were performed as described above.

PC-3 cells were treated with decreasing concentrations of PC1464 (10.0-1.25 μM) for 36 hours and assessed using the Dual Sensor: MitoCasp™ Assay. The results are shown in Table 35, which shows clearly the induction of apoptosis at concentrations as low as 2.5 μM with the greatest effect occurring above 5 μM.

TABLE 35
Apoptosis in PC-3 cells treated with different concentrations of PC1464
for 36 hr measured using the Dual Sensor: MitoCasp ™ Assay

| | | Cells with |
|---|---|---|
| PC1464 | Cells with caspase 3/7 | decreased mitochondrial |
| concentration (μM) | activity (%) | membrane potential (%) |
| Untreated | 9.2 | 1.6 |
| 1.25 | 9.6 | 1.8 |
| 2.5 | 15.1 | 3.4 |
| 5.0 | 57.1 | 28.7 |
| 10.0 | 59.6 | 43.2 |

### Example 27

### Treatment of Breast Cancer (MDA-MB-231) Cells with PC1464

MDA-MB-231 breast cancer cells were treated with PC1464 as previously described. Apoptosis assays were performed as described above.

MDA-MB-231 cells were treated with decreasing concentrations of PC1464 (10.0-1.25 μM) for 35 hours and assessed using the Dual Sensor: MitoCasp™ Assay. The results are shown in Table 36, which shows clearly the induction of apoptosis at concentrations as low as 2.5 μM with the greatest effect occurring above 5 μM.

TABLE 36
Apoptosis in MDA-MB-231 cells treated with different concentrations of
PC1464 for 35 hr measured using the Dual Sensor: MitoCasp ™ Assay

| | | Cells with |
|---|---|---|
| PC1464 | Cells with caspase 3/7 | decreased mitochondrial |
| concentration (μM) | activity (%) | membrane potential (%) |
| Untreated | 20.5 | 5.9 |
| 1.25 | 16.7 | 15.6 |
| 2.5 | 32.4 | 21.2 |
| 5.0 | 53.0 | 32.8 |
| 10.0 | 55.7 | 39.6 |

### Example 28

### Treatment of Fibroblast (CRL-2552) Cells with PC1464

Normal non-cancerous CRL-2522 fibroblast cells were treated with PC1464 as previously described. Apoptosis assays were carried out as described above.

CRL-2522 cells were treated with decreasing concentrations of PC1464 (10.0-1.25 μM) for 36 hours and assessed using the Dual Sensor: MitoCasp™ Assay. The results are shown in Table 37. There was a slight increase in CRL-2522 treated cells with Caspase 3/7 activity and decreased mitochondrial membrane potential compared to untreated CRL-2522 cells, however such increases are expected after extended periods of culture.

TABLE 37
Apoptosis in CRL-2522 cells treated with different concentrations of
PC1464 for 36 hr measured using the Dual Sensor: MitoCasp ™ Assay

| | | Cells with |
|---|---|---|
| PC1464 | Cells with caspase 3/7 | decreased mitochondrial |
| concentration (μM) | activity (%) | membrane potential (%) |
| Untreated | 1.9 | 1.4 |
| 1.25 | 1.7 | 1.7 |
| 2.5 | 3.2 | 1.4 |
| 5.0 | 14.4 | 1.3 |
| 10.0 | 19.9 | 2.2 |

### Example 29

### Treatment of Breast Cancer (MDA-MB-231) Cells with Various Saponins

MDA-MB-231 cells were treated with 5.0 μM of saponins PC1422, PC1448, PC1464, PC1526 and PC1596 for 36 hours and assessed using the Dual Sensor: MitoCasp™ Assay. The results are shown in Table 38, which shows clearly the induction of apoptosis for all saponin treated cells compared to untreated cells with saponin PC1596 showing the highest amount of apoptosis.

TABLE 38
Apoptosis in MDA-MB-231 cells treated with 5.0 μM of saponins
PC1422, PC1448, PC1464, PC1526 and PC1596 for 36 hours
measured using the Dual Sensor: MitoCasp ™ Assay

| Saponin | Cells with caspase 3/7 | Cells with decreased mitochondrial |
|---|---|---|
| (5.0 μM) | activity (%) | membrane potential (%) |
| Untreated | 10.0 | 2.2 |
| PC1422 | 41.2 | 21.7 |
| PC1448 | 12.8 | 5.3 |
| PC1464 | 39.9 | 13.7 |
| PC1526 | 60.8 | 15.4 |

### Example 30

### Treatment of Human Colon Cancer Cells (HT-29) with Various Saponins

The HT-29 human colon cancer cell line (ATCC #HTB-38) was cultured using standard methods for HT-29 cancer cells known in the art.

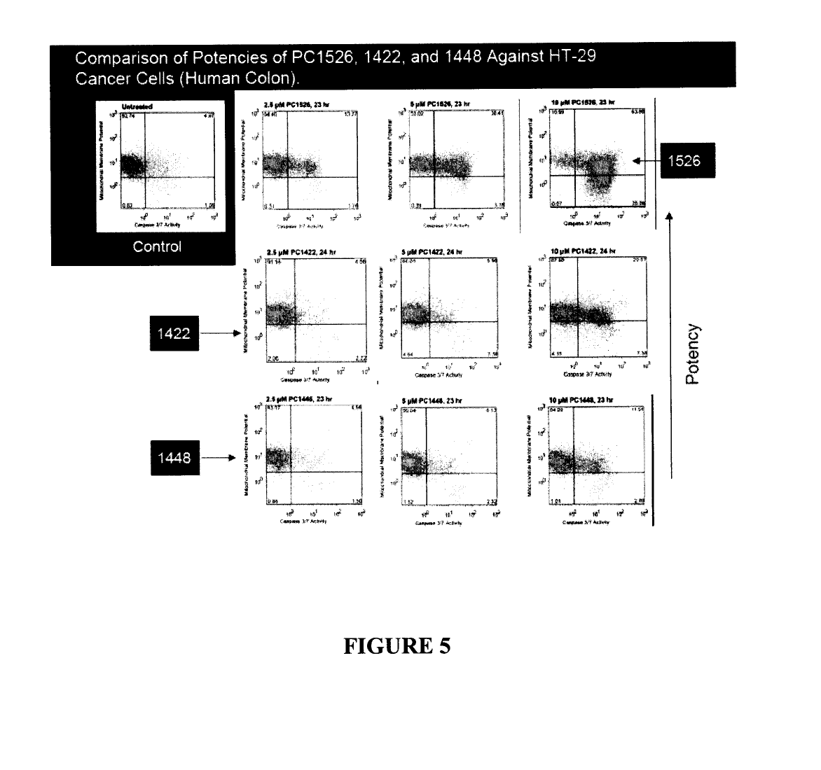

HT-29 cancer cells were treated with 2.5 μM, 5.0 μM and 10 μM of PC1526 or PC1448 for 23 hours, or with 2.5 μM, 5.0 μM and 10 μM PC1422 for 24 hours and assessed using the Dual Sensor: MitoCasp™ Assay. The results are provided in FIG. 5, which show an increase in caspase 3/7 activity (compared to untreated cells) and indicates an induction of apoptosis in HT-29 cells treated with either PC1526, PC1448 or PC1422.

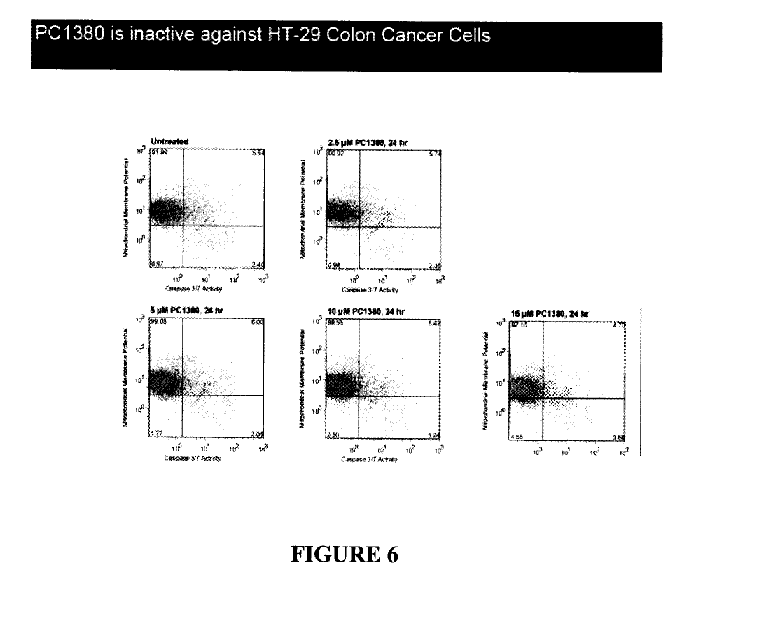

HT-29 cancer cells were treated with 2.5 μM, 5.0 μM, 10 μM and 15 μM of PC1380 for 24 hours and assessed using the Dual Sensor: MitoCasp™ Assay. The results are provided in FIG. 6 and shows that PC1380 does not induce apoptosis in HT-29 colon cancer cells.

### Example 31

### Treatment of Human Colon Cancer Cells (WiDr) with PC1526

The WiDr human colon cancer cell line (ATCC #CCL-218™) was cultured using standard methods for WiDr cancer cells known in the art.

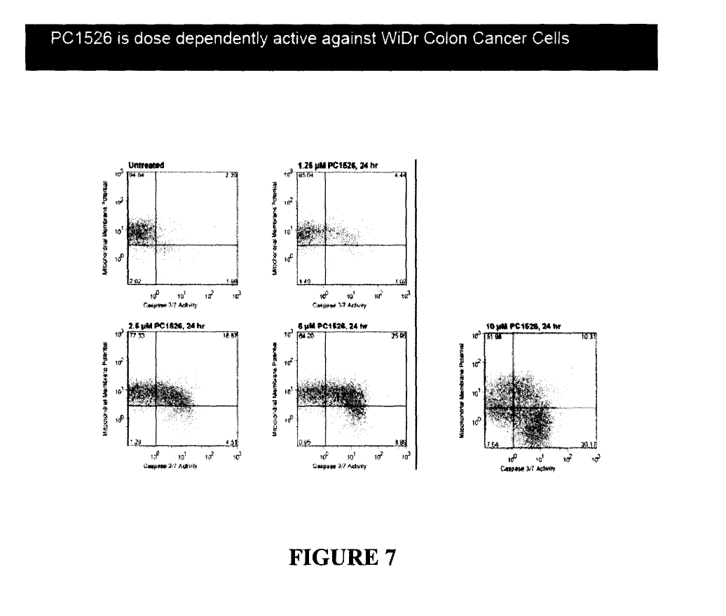

WiDr colon cancer cells were treated with 1.25 μM, 2.5 μM, 5.0 μM, and 10 μM of PC1526 for 24 hours and assessed using Dual Sensor: MitoCasp™ Assay. The results are shown in FIG. 7, which shows that PC1526 induces apoptosis in WiDr colon cancer cells (compared to untreated cells).

### Example 32

### Treatment of Human Colon Cancer Cells (WiDr) with PC1448

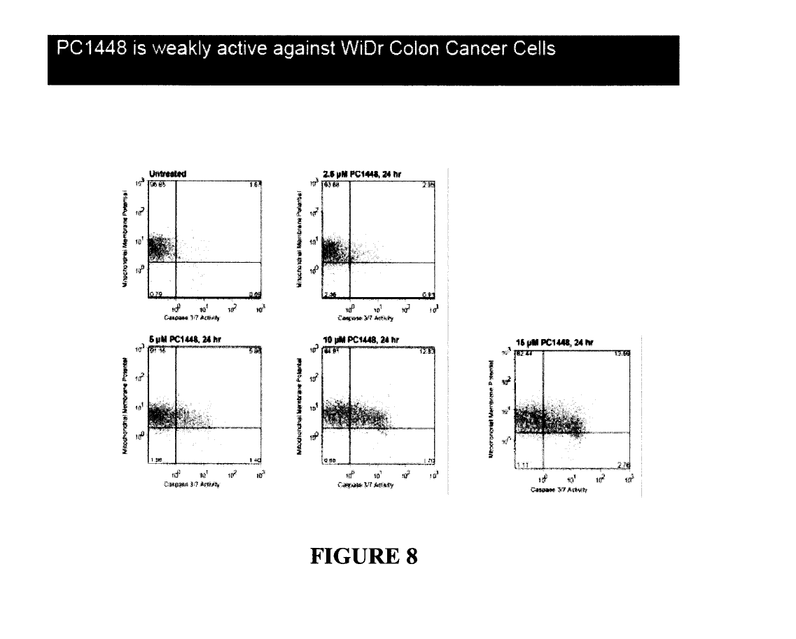

WiDr colon cancer cells were treated with 2.5 μM, 5.0 μM, 10 μM, and 15 μM of PC1448 for 24 hours and assessed using Dual Sensor: MitoCasp™ Assay. The results are provided in FIG. 8 and shows that PC14481 induces apoptosis in WiDr colon cancer cells (compared to untreated cells).

### Example 33

### Treatment of Human Colon Cancer Cells with PC1596

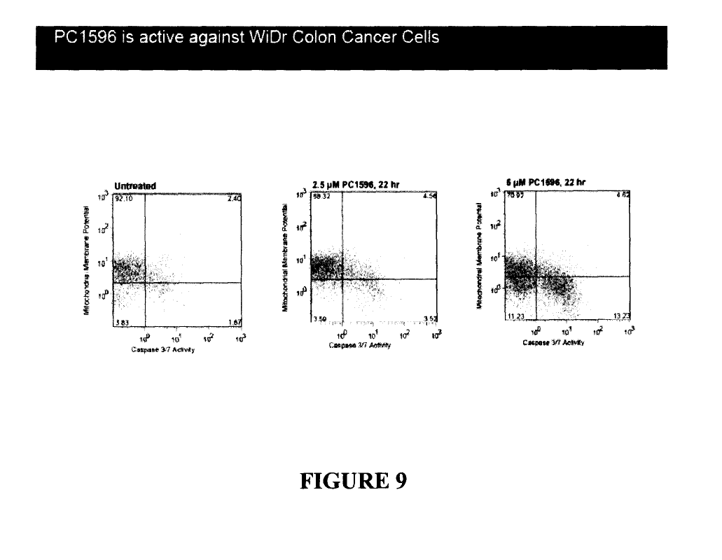

WiDr colon cancer cells were treated with 2.5 μM, and 5.0 μM of PC1596 for 22 hours and assessed using Dual Sensor: MitoCasp™ Assay. The results are presented in FIG. 9. PC1596 induces apoptosis in WiDr colon cancer cells (compared to untreated cells).

### Example 34

### Treatment of Breast Cancer Cells with Calendula Saponin

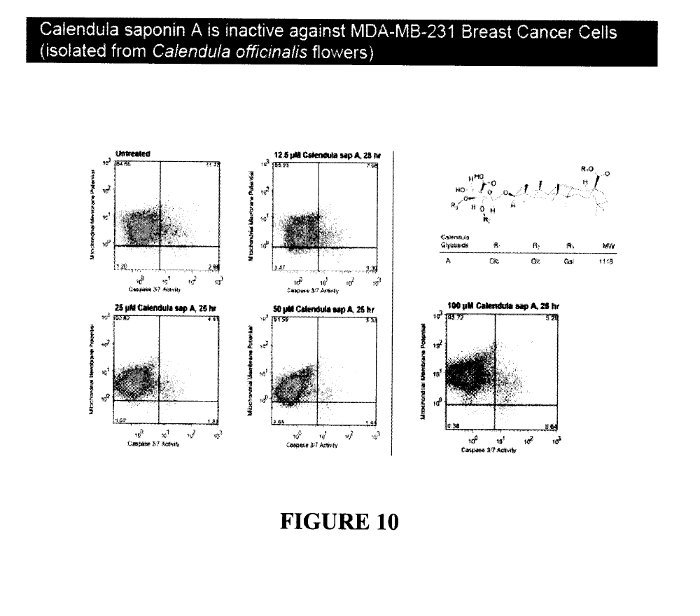

MDA-MB-231 breast cancer cells were treated with 12.5 μM, 25 μM, 50 μM and 100 μM for 25 hours with oleanolic bisdesmoside isolated from Calendula flowers (Calendula officinalis) and assessed using Dual Sensor: MitoCasp™ Assay. The data obtained as shown in FIG. 10 demonstrate that the concentrations of Calenulda saponin tested do not induce apoptosis in the MDA-MB-231 cell line.

### Example 35

### Treatment of Prostate Cancer Cells with PC1688

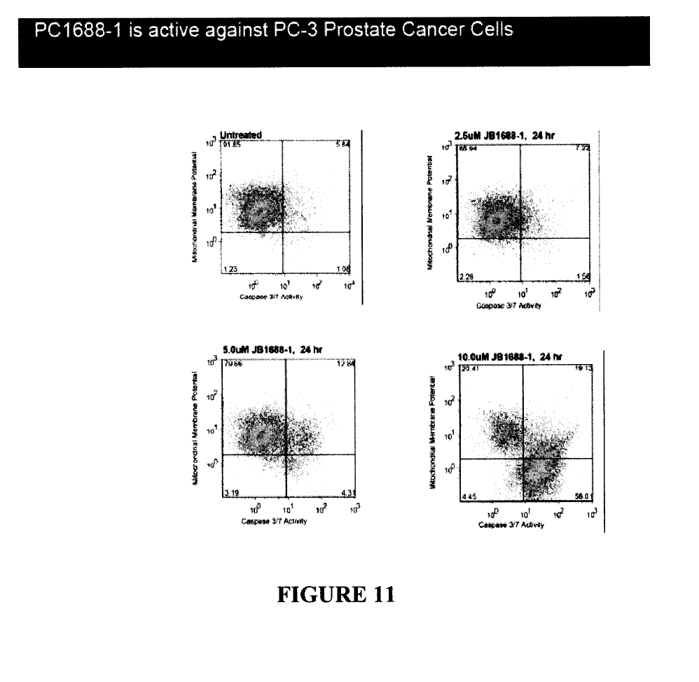

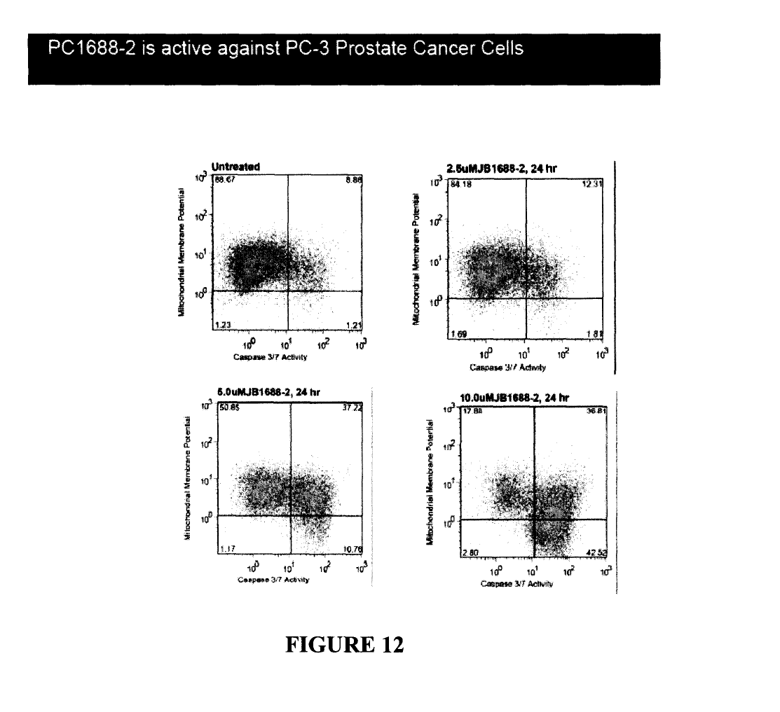

PC-3 prostate cancer cells were treated with 2.5 μM, 5 μM and 10 μM of PC1688-1 or PC1688-2 for 24 hours and assessed using Dual Sensor: MitoCasp™ Assay. The results are shown in FIG. 11 (PC1688-1) and FIG. 12 (PC1688-2), and demonstrate that PC1688-1 and PC1688-2 both induce apoptosis in PC-3 prostate cancer cells.

### Example 36

### Treatment of Breast Cancer Cells with PC1688-1

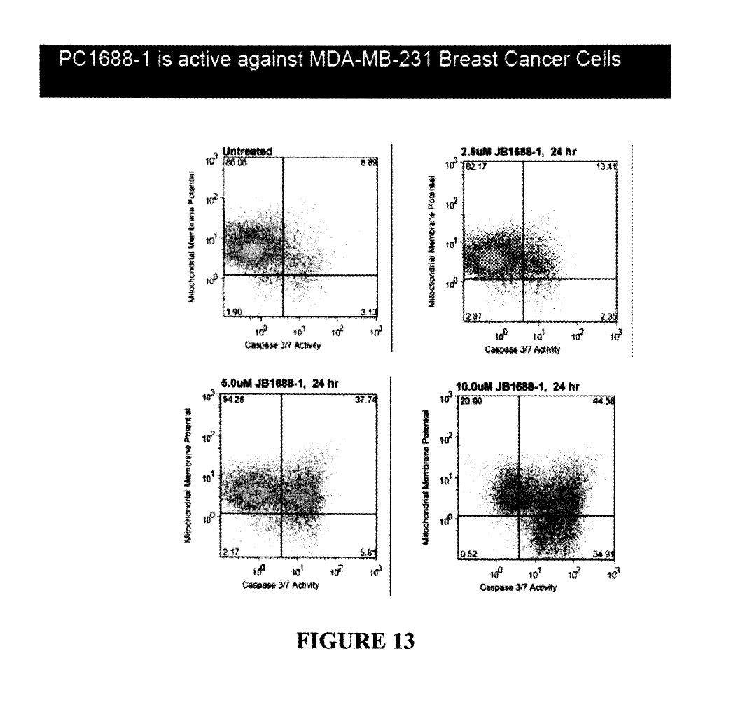

MDA-MB-231 Breast Cancer Cells were treated with 2.5 μM, 5 μM and 10 μM of PC1688-1 for 24 hours and assessed using Dual Sensor: MitoCasp™ Assay. The results are presented in FIG. 13, demonstrating that PC1688-1 induces apoptosis in MDA-MB-231 cells.

### Example 37

### Treatment of Prostate Cancer Cells with PC1526

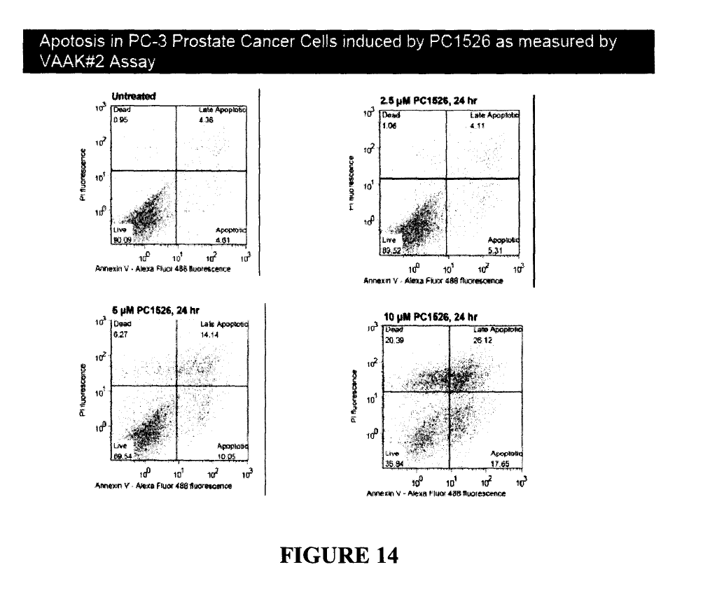

PC-3 prostate cancer cells were treated with 2.5 μM, 5 μM and 10 μM of PC1526 for 24 hours and assessed using the Vybrant® Apoptosis Assay Kit #2. The results are shown in FIG. 14. PC1512 induces apoptosis in PC-3 cells (compared to untreated cells).

### Example 38

### Treatment of Breast Cancer Cells with PC1526

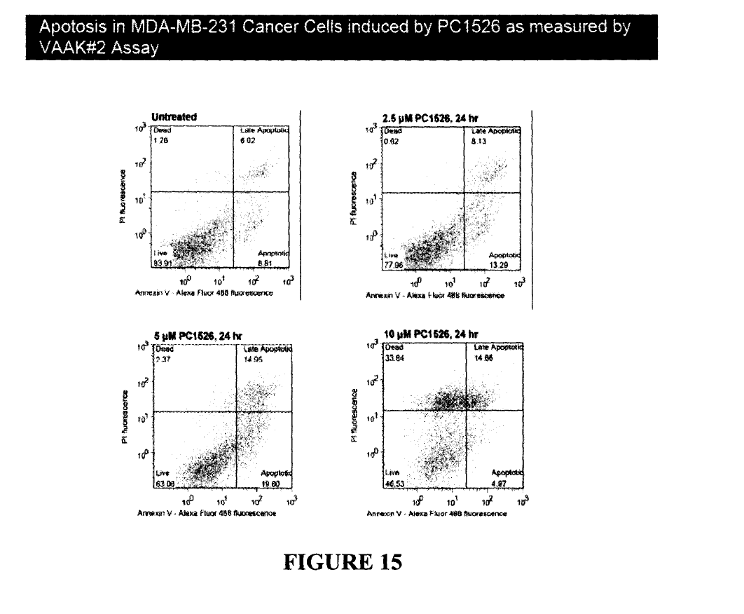

MDA-MB-231 cancer cells were treated with 2.5 μM, 5 μM and 10 μM of PC1526 for 24 hours and assessed using the Vybrant® Apoptosis Assay Kit #2. The results are shown in FIG. 15, which shows that PC1512 is inducing apoptosis in MDA-MB-231 cells (compared to untreated cells).

### Example 39

### Treatment of Breast Cancer Cells (MCF-7) with Various Saponins

The MCF-7 human breast cancer cell line (ATCC #HTB-22™) was cultured using standard methods for MCF-7 cancer cells known in the art.

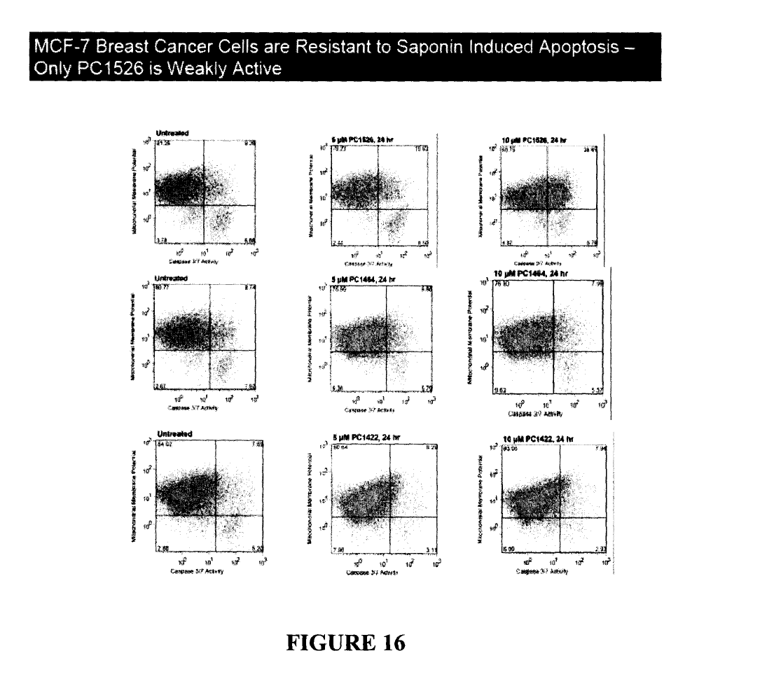

MCF-7 cells were treated with 5.0 μM and 10 μM of saponins PC1526, PC1464 and PC1422 for 24 hours and assessed using Dual Sensor: MitoCasp™ Assay. The results are shown in FIG. 16, which shows that PC1464 and PC1422 at concentrations of 5 and 10 μM do not induce apoptosis in MCF-7 breast cancer cells. PC1526 does not induce apoptosis at a concentration of 5 μM. PC1526 induces apoptosis at a concentration of 10 μM.

### Example 40

### Haemolytic Properties of Selected Saponins

The haemolytic properties of the various saponins that have been purified to more than 75% have been examined and are provided in Table 39. Haemolytic properties are not directly related to anti-cancer or apoptosis inducing properties of saponins but may be related to toxicity and utility of the saponins for preparation of injectable formulations. Saponins most suitable for injectable formulation will be less haemolytic. QS21, a purified Quillaja saponaria saponin with known haemolytic activity is used as reference.

TABLE 39

| | | Order of Apoptosis | |
|---|---|---|---|
| Entry | Saponin | Potency* | HD50 (μM) |
| 1 | PC-1448 | 7 | 13.8 |
| 2 | PC-1596 | 2 | 14.8 |
| 3 | PC-1464 | 4 | 17.5 |
| 4 | PC-1422 | 4 | 43.8 |
| 5 | PC-1526 | 1 | 51.4 |
| 6 | PC-1688-1 | 4 | 55.1 |
| 7 | PC-1688-2 | 3 | 87.7 |
| Ref. | QS21 | | 7.5 |

*Apoptosis inducing activity, based on responses observed in PC-3, MDA-MB-231, WiDr, and HT-29 Cells; 1 = most active.

All citations are hereby incorporated by reference.

The present invention has been described with regard to one or more embodiments. However, it will be apparent to persons skilled in the art that a number of variations and modifications can be made without departing from the scope of the invention as defined in the claims.

### REFERENCES

Atopinka, L. N., et al., Cytotoxicity of natural ginseng glycosides and semisynthetic analogues. Planta Medica 65:30-34, (1999).
Baslevich, J., Bishop, G., Ramiriez-Erosa, I., 2006. Analysis of bisdesmosidic saponins in Saponaria vaccaria L. via LC-MS-PDA. Phytochemical Analysis 17:414-423
Barthomeuf, C., et al., Inhibition of HUVEC tubulogenesis by hederacolchiside-A1 is associated with plasma membrane cholesterol sequestration and activation of the Ha-Ras/MEK/ERK cascade. Cancer Chemother Pharmacol 54:432-440, (2004).
Budihardjo, I., et al., Biochemical pathways of caspase activation during apoptosis. Annu. Rev. Cell Dev. Biol. 15:269-90, (1999)
Cailleau R., et al., Breast tumor cell lines from pleural effusions. J. Natl. Cancer Inst. 53: 661-674, 1974.
Chaing, Y-M., et al., Cytotoxic triterpenes from the aerial roots of Ficus microcarpa, Phytochemistry 66:495-501, (2005)
Chang, C-I., et al., Three new oleanane-type triterpenes from Ludwigia octovalvis with cytotoxic activity against two human cancer cell lines, J. Nat. Prod. 67:91-93, (2004).
Chang, Y. S., et al., Panax ginseng: A role in cancer therapy? Integrative cancer Therapies 2:13-33, (2003).
Chow, L. M. C., et al., Antiproliferative activity of the extract of Gleditsia sinensis fruit on human solid tumour cell lines, Chemotherapy 48:303-308, (2002)
Cichewicz, R. H., et al., Chemistry, biological activity, and chemotherapeutic potential of betulinic caid for the prevention and treatment of cancer and HIV infection. Medicinal Research Reviews 24:90-114, (2004)
Cloffi, G., et al., Cytotoxic saponins from Schefflera fagueti. Planta Med 69:750-756, (2003)
Debatin, K. M., Activation of apoptosis pathways by anticancer drugs. Adv Exp Med Biol 457: 237-44, (1999).
Ellington, A. A., et al., Induction of macroautophagy in human colon cancer cells by soybean B-group triterpenoid saponins, Carcinogenesis 26:159-167, (2005)
Feng, X-Z., et al., Three new triterpenoid saponins from Ixeris sonchifolia and their cytotoxic activity. Plant Med 69:1036-1040, (2003)
Francis, G., et al., The biological action of saponins in animal systems: a review. Brit. J. Nutrition. 88:587-605, (2002).
Gaidi, G., et al., Saponins-mediated potentiation of cisplatin accumulation and cytotoxicity in human colon cancer cells. Plant Med 68:70-72, (2002)
Gross, S. C., et al. Antineoplastic activity of Solidago virgaurea on prostatic tumor cells in an SCID mouse model, Nutrition and Cancer 43: 76-81, (2002)
Gulbins, E., et al., Role of mitochondria in apoptosis, Exp Physiol 88:85-90, (2003)
Hacker, G., The morphology of apoptosis. Cell Tissue Res. 301(1):5-17, (2000)

Haddad, M., Induction of apoptosis in a leukemia cell line by triperpene saponins from Albizia adianthifolia. Bioorganic & Medicinal Chemistry 12:4725-4734, (2004)
Hengartner, M. O., The biochemistry of apopotsis. Nature 427:770-8., (2000).
Huang, Y-T., et al., Investigation of oubain-induced anticancer effect in human androgen-independent prostate cancer PC-3 cells, Biochemical Pharmacology 67:727-733, (2004).
Hsu, Y-L., et al., Effect of saikosaponin, a triterpene saponin, on apoptosis in lymphocytes: association with c-myc, p53 and bcl-2 mRNA. British J Pharmacology 131:1285-1293, (2000)
Hsu, Y-L., et al., The proliferative inhibition and apoptotic mechanism of Saikosaponin D in human non-small cell lung cancer A549 cells. Life Sciences 75:1231-1242, (2004)
Hwang, S. J., et al., Diol- and triol-type ginseng saponins potentiate the apoptosis of NIH3T3 cells exposed to methyl methanesulfonate, Toxicology and Applied Pharmacology 181:192-202, (2002)
Kaighn, M. E., et al., Establishment and characterization of a human prostatic carcinoma cell line (PC-3), Invest. Urol 17:16-23, (1979).
Kaufmann, S. H., et al., Induction of apoptosis by cancer chemotherpay. Exp Cell Research 256:42-49, (2000).
Kashiwada, Y., et al., Antitumor agents. 180. Chemical studies and cytotoxic evaluation of cumingianosides and cumindyoside A, antileukemic triterpene glucosides with a 14,18-cycloapotirucallane skeleton, J. Nat. Prod. 60:1105-1114, (1997)
Kim, H-S., et al., Effects of ginsenosides Rg3 and Rh2 on the proliferation of prostate cancer cells, Arch Pham Res 27:429-435, (2004)
Kroemer, G., et al., Mitochondrial control of cell death. Nat Med 6:513-9, (2000)
Lin, H., et al., Involvement of Cdk5/p25 in digoxin-triggered prostate cancer cell apoptosis, J. Biol. Chem. 28:29302-29307, (2004)
Lin, J., et al., Effects of astragali radix on the growth of different cancer cell lines, World J. Gastroenterol. 9:670-673., (2003)
López-Lāzaro, M., et al., Anti-tumour activity of Digitalis purpurea L. subsp. Heywoodii. Planta Med 69:7-1-704, (2003).
López-Lāzaro, M., et al., Digitoxin inhibits the growth of cancer cell lines at concentrations commonly found in cardiac patients, J. Nat. Prod. 68:1642-1645, (2005)
Marquina, S., et al., Bioactive oleanolic acid saponins and other constituents from the roots of Viguiera decurrens, Phytochemistry 56:93-97, (2001)
Martin, S. J. et al. Early Redistribution of Plasma Membrane Phosphatidylserine Is a General Feature of Apoptosis Regardless of the Initiating Stimulus: Inhibition by Overexpression of Bcl-2 and Abl. J. Exp. Med. 182: 1545-1556 (1995)
Mimaki, Y., et al., Triterpene glycosides from the roots of Sanguisorba officinalis, Phytochemistry 57:773-779, (2001)
Mimaki, Y., et al., Enterolosaponins A and B, novel triterpene bisdesmosidics from Enterolobium contortisssiliquum, and evaluation for their macrophage-oriented cytotoxic activity, Bioorganic & Medical Chemistry Letters 13:623-627, (2003)
Mimaki, Y., et al., Triterpene saponins from the roots Clematis chinensis, J. Nat. Prod. 67:1511-1516, (2004).
Nagaya, H., et al., Cytotoxic triterpenes from Cleome Africana, Phytochemistry 44:1115-1119, (1997).
Park, D., et al. Induction of apoptosis and inhibition of telomerase activity by aqueous extract from Platycodon grandiflorum in human lung carcinoma cells. Pharmacological Research 51(5):437-43 (2005)
Plohmann, B., et al., Immunomodulatory and antitumoral effects of triterpinoid saponins. Pharmazie 52:953-57, (1997)
Prakash Chaturveldula, V. S., et al. New cytotoxic oleanane saponins from the infructescences of Polyscias amplifolia from the Madigascar rainforest. Plant Med 69:440-444, (2003)
Shao, Y., et al., Triterpenoid saponins from Aster lingulatus, Phytochemistry 44:337-340, (1997).
Sparg, S. G., et al., Biological activities and distribution of plant saponins. J. Ethnopharmacology 94:219-43, (2004).
Sung, M. K., et al., Effect of soybean saponins and Gypsophilia saponin on morphology of colon carcinoma cells in culture, Fd Chem Toxic 33:357-366, (1995).
Tian, J-K., et al., New cytotoxic saponins from Lysimachia davurica Ledeb. J Integrative Plant Biology 48:232-235, (2006)
Wang, F., et al., Role of mitochondria and mitochondrial cyctochrome C in tubeimoside-I-mediated apoptosis of human cervical carcinoma HeLa cell line. Cancer Chemother Pharmacol 57:389-399, (2006).
Wang, Y., et al., Exploration of the correlation between the structure, hemolytic activity, and cytotoxicity of steroid saponins. Biorganic and Medicinal Chemistry, 2007, (in press).
Wargovich, M. J., Colon cancer chemoprevention with ginseng and other botanicals, J. Korean Med. Sci 16(Suppl) S81-6, (2001)
Wu, C-A., et al., Induction of cell death by saponin and antigen delivery, Pharmaceutical Research 21:271-277, (2004)
Wu, C-C., et al., Pristimerin induces caspase-dependent apoptosis in MDA-MB-231 cells via direct effects on mitochondria, Mol Cancer Ther. 4: 1277-85, (2005).
Xiao, K., et al., A cytotoxic triterpine saponin from the root bark of Aralia dasyphylla. J. Nat. Prod. 62:1030-1032, (1999)
Yanamandra, N., et al., Triterpenoids from Glycine max decrease invasiveness and induce caspase-mediated cell death in human SNB19 glioma cells. Clinical & Experimental Metastatis 20:375-383, (2003)
Yesiladaa, E., et al., Effects of triterpine saponins from Astragalus species on in vitro cyctokine release. J. Ethnopharmacology 96:71-77, (2005).
Yui, S., et al., Macrophage-oriented activity of novel triterpene saponins extracted from roots of Seuridaca inappendiculata, International Immunopharmacology 1:1989-2000, (2001).
Yui, S., et al., Characterization of the growth-inhibitory and apoptosis-inducing activities of a triterpenoid saponin securioside B against BAC1.2F5 macrophages. Mediators of Inflammation 12:157-166, (2003).
Zhong, L., et al., Structure-activity relationship of saponins from Gleditsia sinensis in cytotoxicity and induction of apoptosis. Plant Med 70:797-802, (2004).

1-17. (canceled)

18. A bisdesmosidic saponin having a molecular weight selected from the group of 1526, 1596 and1688.

19. The bisdesmosidic saponin of claim 18 wherein the molecular weight is 1688.

20. A pharmaceutical composition comprising at least one bisdesmosidic saponin having a molecular weight selected from the group of 1526, 1596 and 1688, and a pharmaceutically acceptable carrier.

21. The pharmaceutical composition of claim 20 wherein the bisdesmosidic saponin has a molecular weight of 1688.

22. The pharmaceutical composition of claim 21 wherein the bisdesmosidic saponin having a molecular weight of 1688 is present in more than one isomeric form.

23. The pharmaceutical composition of claim 21 wherein the bisdesmosidic saponin having a molecular weight of 1688 is present in one isomeric form.

24. The pharmaceutical composition for use in treating breast cancer, colon cancer or prostate cancer, said composition comprising at least one apoptosis inducing bisdesmosidic saponin having a molecular weight selected from the group of 1448, 1422, 1526, 1596 and 1688, and a pharmaceutically acceptable carrier.

25. A method of treating breast cancer, colon cancer or prostate cancer comprising, administering a therapeutic amount of at least one bisdesmosidic saponin having a molecular weight selected from the group of 1448, 1422, 1526, 1596 and 1688 to a patient in need thereof.

26. The method of claim 25 wherein the bisdesmosidic saponin has a molecular weight of 1688.

27. A method of inducing apoptosis in a breast cancer, colon cancer or prostate cancer cell, comprising exposing the cell to a bisdesmosidic saponin having a molecular weight selected from the group consisting of 1448, 1422, 1526, 1596 and 1688.

28. The method of claim 27 wherein the bisdesmosidic saponin has a molecular weight of 1688.

29. A method of producing an isolated bisdesmosidic saponin composition from seed of Saponaria vaccaria comprising:
a) milling seed of Saponaria vaccaria to produce a milled seed;
b) treating the milled seed with a first solvent to produce a saponin mixture;
c) cooling the saponin mixture to produce a precipitate comprising a composition of bisdesmosidic saponins; and
d) recovering the precipitate comprising the isolated bisdesmosidic saponin composition.

30. The method of claim 29 wherein after the step of treating and before the step of cooling, a second solvent is added to the saponin mixture.

31. A method of producing an isolated bisdesmosidic saponin composition from root of Saponaria vaccinia comprising:
a) pulverizing dried root of Saponaria vaccaria to produce a pulverized root;
b) treating the pulverized root with a first solvent to produce a saponin mixture;
c) cooling the saponin mixture to produce a precipitate comprising a composition of bisdesmosidic saponins; and
d) recovering the precipitate comprising the isolated bisdesmosidic saponin composition.

32. The method of claim 31 wherein after the step of treating and before the step of cooling, a second solvent is added to the saponin mixture.

33. A method of producing an isolated bisdesmosidic saponin composition from a tissue culture or a cell culture of Saponaria vaccinia comprising:
a) pulverizing the tissue culture or the cell culture of Saponaria vaccaria to produce a pulverized tissue culture or cell culture;
b) treating the pulverized tissue culture or cell culture with a first solvent to produce a saponin mixture;
c) cooling the saponin mixture to produce a precipitate comprising a composition of bisdesmosidic saponins; and
d) recovering the precipitate comprising the isolated bisdesmosidic saponin composition.

34. The method of claim 33 wherein after the step of treating and before the step of cooling, a second solvent is added to the saponin mixture.

35. A method of purifying a bisdesmosidic saponin from Saponaria vaccinia comprising:
a) applying the isolated bisdesmosidic saponin composition of claims 29 to a chromatographic column; and
b) recovering the purified bisdesmosidic saponin from the chromatographic column.

36. A method of purifying a bisdesmosidic saponin from Saponaria vaccinia comprising:
a) applying the isolated bisdesmosidic saponin composition of claims 31 to a chromatographic column; and
b) recovering the purified bisdesmosidic saponin from the chromatographic column.

37. A method of purifying a bisdesmosidic saponin from Saponaria vaccinia comprising:
a) applying the isolated bisdesmosidic saponin composition of claims 33 to a chromatographic column; and
b) recovering the purified bisdesmosidic saponin from the chromatographic column.
